SEQUENCE LISTING (1 sequence)

SEQ ID NO: 1 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

(19) United States
(12) Patent Application Publication
(10) Pub. No.: US 2023/0398176 A1
(43) Pub. Date: Dec. 14, 2023

(54) COMPOSITIONS AND METHODS FOR IDENTIFYING AND TREATING MICROPARTICLE-ASSOCIATED DISEASES AND CONDITIONS

(71) Applicants: BioAegis Therapeutics Inc., Morristown, NJ (US); University of Maryland, Baltimore, Baltimore, MD (US)

(72) Inventors: Susan L. Levinson, Morristown, NJ (US); Stephen R. Thom, Baltimore, MD (US); Mark J. DiNubile, Morristown, NJ (US); Thomas P. Stossel, Morristown, NJ (US)

(73) Assignees: BioAegis Therapeutics Inc., Morristown, NJ (US); University of Maryland, Baltimore, Baltimore, MD (US)

(21) Appl. No.: 18/246,255

(22) PCT Filed: Sep. 23, 2021
(86) PCT No.: PCT/US2021/071559

§ 371 Date: Mar. 22, 2023

Related U.S. Application Data
(60) Provisional application No. 63/082,277, filed on Sep. 23, 2020.
(60) Provisional application No. 63/148,808, filed on Feb. 12, 2021.

(51) Int. Cl. A61K 38/16; G01N 33/68; A61P 43/00

(52) U.S. Cl. CPC A61K 38/16; G01N 33/6863; G01N 33/6893; A61P 43/00; G01N 2800/52; G01N 2800/709

(57) ABSTRACT

The present invention relates to compositions and methods for identifying and treating signature MP-associated diseases and conditions in subjects. In particular, treatment methods of the invention include administering a gelsolin agent to produce a therapeutic effect against a signature MP-associated disease or condition in a subject.

### RELATED APPLICATIONS

This application claims benefit under 35 U.S.C. § 119(e) of U.S. Provisional application Ser. No. 63/082,277 filed Sep. 23, 2020 and U.S. Provisional application Ser. No. 63/148,808 filed Feb. 12, 2021, the disclosure of each which is incorporated by reference herein in its entirety.

### GOVERNMENT INTEREST

This invention was made with government support under N00014-20-1-2641 and N000-I4-16-1-2868 awarded by the U.S. Office of Naval Research. The government has certain rights in the invention.

### FIELD OF THE INVENTION

The invention, in some aspects, relates to compositions and methods for identifying and treating a signature microparticle-associated disease or condition.

### BACKGROUND OF THE INVENTION

Inert gases inhaled while breathing are taken up by tissues in proportion to the ambient pressure. When pressure is reduced, some of the gas released from tissues forms bubbles due to the presence of gas cavitation nuclei [see for example Ljubkovic M et al., Med Sci Sports Exerc 43: 990-995, 2011; Ljubkovic M et al., J Appl Physiol 109: 1670-1674, 2010; Lu C-H et al., Arch Biochem Biophys 529: 146-156, 2013]. The central place of bubbles as an inciting agent for decompression sickness (DCS) is widely accepted. However, because most decompression procedures generate asymptomatic blood-borne bubbles based on ultrasound studies, additional factors precipitating DCS are under investigation [see for example Madden D et al., Med Set Sports Ewe 46: 1928-1935, 2014; Madden D et al., Eur J Appl Physiol 114: 1955-1961, 2014; Madden L A et al., Aviat Space Environ Med 81: 41-51, 2010]. High pressure gases also trigger formation of small vesicles called microparticles (MPs) [see for example Miles L A et al., J Neurosci 26: 13017-13024, 2006]. The number of blood-borne MPs doubles in mice and humans exposed to high gas pressure and rise further after decompression [see for example Moroianu J et al., PNAS 90: 3815-3819, 1993; Ordija C M et al., Am J Physiol Lung Cell Mol Physiol 312: L1018-L1028, 2017; Osborn T M et al., Am J Physiol Cell Physiol 292: C1323-1330, 2007; Osborn T M et al., Arthritis Res Ther 10: R117, 2008; Overmyer K A et al., medRxiv https://doi.org/10.1101/2020.07.17.20156513: 2020; Pardridge W M et al., J Cereb Blood Flow Metab 9: 675-680, 1989; Peddada N et al., Med Hypotheses 778: 203-210, 2012; Philip R B. UnderseaBiomedRes 1: 117-150, 1974; Piktel E et al., Int J Mol Sci 19: 2516: 1-33, 2018; Pontier J M et al., Appl Physiol Nutr Metab 37: 1-5, 2012]. Murine studies indicate that MPs play a role in high pressure gas pathophysiology and possibly gas bubble nucleation [see for example Piktel E et al., Int J Mol Sci 19: 2516: 1-33, 2018; Por S B et al., J Histochem Cytochem 39: 981-985, 1991; Rothmeier A S et al., J Clin Invest 125: 1471-1484, 2015; Smalheiser N R. Mol Biol Cell 7: 1003-1014, 1996).

Decompression sickness is a condition that results from the dissolution of gas bubbles (usually nitrogen) into tissues of an individual. The dissolution is generally caused when the individual is exposed to a relatively rapid decrease in environmental pressure.

Decompression sickness can be caused by a variety of factors, but most common are: rapid ascent from a deep scuba dive (generally depths greater than about 10 meters or about 33 feet); rapid ascent in an airplane with an unpressurized cabin; rapid loss of pressure in an airplane (e.g., loss of cabin pressure at high altitudes); sub aqueous tunnel work (e.g., caisson work); inadequate pressurization/denitrogenation when flying; and flying to a high altitude too soon after scuba diving.

Of these factors, the most common cause of decompression sickness occurs from scuba divers ascending too quickly from a relatively deep dive. During deep dives, divers are exposed to higher and higher ambient pressures as they descend. Because of the higher pressures, the inert gases such as nitrogen and helium, which are included in the breathing gases of the diver, are adsorbed into the tissues of the body in higher concentrations than normal. When a diver ascends from the dive, the ambient pressure is reduced causing the absorbed gases to come back out of solution and form “micro bubbles” in the blood. If the ascent is done slowly, the micro bubbles will safely leave the body through the lungs, i.e., expiration. However, during a rapid ascent not all of the micro bubbles leave the body, thereby resulting in decompression sickness.

The primary treatment for decompression sickness is hyperbaric oxygen therapy. Hyperbaric oxygen therapy is a mode of therapy in which the patient breathes 100% oxygen at pressures greater than normal atmospheric pressure. Generally, hyperbaric oxygen therapy involves the systemic delivery of oxygen at levels 2-3 times greater than atmospheric pressure. The oxygen under pressure reduces the micro bubble size in the patient, creating a pressure gradient for nitrogen gas expulsion and forcing oxygen into ischemic tissue.

Hyperbaric oxygen therapy is also disadvantageous in that in smaller, single occupancy chambers, the patient is left in relative isolation. This is a special concern with patients suffering from a severe case of decompression sickness or with patients who are suffering from conditions in addition to decompression sickness that require medical personnel to be in close proximity with the patient (e.g., having a wound that requires suturing). The small chambers act as a barrier, preventing the medical personnel from closely monitoring the patient and preventing the medical personnel from administering medical services while the patient is receiving HBO therapy. Other treatments for decompression sickness are also known, such as 100% oxygen at atmospheric pressure by mask, dextran and standard replacement fluids to correct hypovolemia. These treatments are not fully effective in isolation. Rather, these alternative treatments are adjunctive therapies, i.e., treatments used together with the primary treatment to assist the primary therapy.

Inflammatory responses play a role in the pathophysiology of decompression sickness [see for example Bigley N J et al., J Interferon Cytokine Res 28: 55-63, 2008; Khatri N et al., J Diab Res 2014: 152075: 2014; Miles L A et al., J Neurosci 26: 13017-13024, 2006; Overmyer K A et al., medRxiv https://doi.org/10.1101/2020.07.17.20156513: 2020]. Plasma gelsolin (pGSN) is a 84 kDa secreted isoform of a cytoplasmic actin-binding protein [see for example Bucki R et al., Am J Physiol Cell Physiol 299: C1516-1523, 2010]. It depolymerizes circulating filamentous actin (F-actin), binds/sequesters an array of inflammatory agents, and by attaching to microorganisms will accelerate phagocytosis and macrophage bactericidal actions [see for example Brett K D et al., Sci Rep in press: https://doi.org/10.1038/s41598-41019-49924-41591, 2019; Bucki R et al., J Immunol 181: 4936-4944, 2008; Bucki R et al., Biochemistry 44: 9590-9597, 2005; Ljubkovic M et al., J Appl Physiol 109: 1670-1674, 2010; Lu C-H et al., Arch Biochem Biophys 529: 146-156, 2013; Thom S R et al., J Appl Physiol 119: 427-434, 2015; Thom S R. et al., J Biol Chem 292: 18312-18324, 2017].

Inert gases inhaled while breathing are taken up by tissues in proportion to the ambient pressure and when pressure is reduced, some of the gas released from tissues forms bubbles due to the presence of gas cavitation nuclei [see for example D. J. Kwiatkowski D J et al., Nature 323: 455-458, 1986; Thom S R et al., J Appl Physiol (1985) 125: 1339-1348, 2018; Thom S R et al., J Appl Physiol 112: 1268-1278, 2012]. The central place of bubbles as an inciting factor for decompression sickness is widely accepted, yet most decompression procedures generate asymptomatic blood-borne bubbles [see for example Cypryk W et al., Front Immunol 9: 2188, 2018; Kinosian H J et al., Biochemistry 35: 16550-16556, 1996; Lee P S et al., PLoS One 3: e3712, 2008]. High pressure gases also trigger formation of small vesicles called microparticles (MPs) [see for example Philip R B, UnderseaBiomedRes 1: 117-150, 1974]. The number of blood-borne microparticles double in mice and humans exposed to high gas pressure and rise further after decompression [see for example Bohgaki M et al., J Cell Mol Med 15: 141-151, 2011; Lind S E et al., Am Rev Respir Dis 138: 429-434, 1988; Little T et al., Aviat Space Environ Med 79: 87-93, 2008; Ljubkovic M et al., Med Sci Sports Exerc 43: 990-995, 2011; Ordija C M et al., Am J Physiol Lung Cell Mol Physiol 312: L1018-L1028, 2017; Pontier J M et al., Appl Physiol Nutr Metab 37: 1-5, 2012; Por S B et al., J Histochem Cytochem 39: 981-985, 1991; Rothmeier A S et al., J Clin Invest 125: 1471-1484, 2015; Smalheiser N R, Mol Biol Cell 7: 1003-1014, 1996; Thom S R et al., J Appl Physiol (1985) 126: 1006-1014, 2019]. The pathway triggering microparticle formation also activates the NOD-like receptor, pyrin containing 3 (NLRP3) inflammasome responsible for producing mature interleukin (IL)-1β [see for example Philip R B, Undersea Biomed Res 1: 117-150, 1974; Piktel E et al., Int J Mol Sci 19: 2516: 1-33, 2018]. Microparticles produced in response to high pressure contain high amounts of IL-1β, and are the primary factor causing diffuse vascular damage in a murine decompression sickness model [see for example Peddada N et al., Med Hypotheses 778: 203-210, 2012; Pontier J M et al., Appl Physiol Nutr Metab 37: 1-5, 2012]. When these microparticles are purified and injected into naïve mice, they cause the same spectrum of tissue damage as seen in decompressed mice [see for example Pontier J M et al., Appl Physiol Nutr Metab 37: 1-5, 2012; Smalheiser N R, Mol Biol Cell 7: 1003-1014, 1996].

The prior art is deficient in understanding the relationship between the plasma protein gelsolin and the stress imposed by high pressure and decompression as well as methods of treating decompression sickness. The present invention fulfills this longstanding need and desire in the art.

### SUMMARY OF THE INVENTION

According to an aspect of the invention, a method of determining the presence of a signature MP-associated disease or condition in a subject is provided, the method including: (a) detecting in a biological sample obtained from a subject suspected of having a signature MP-associated disease or condition, the presence of microparticles; (b) identifying the detected microparticles as including an IL-1β signature, a lymphocyte antigen 6 complex locus G6D (Ly6G) signature, or a CD66b signature; wherein the identification of the IL-1β, Ly6G, or CD66b signature confirms the presence of the signature MP-associated disease or condition in the subject; (c) selecting a therapeutic regimen for the subject based at least in part on the confirmation of the presence of the signature MP-associated disease or condition in the subject; and (d) administering the selected therapeutic regimen to the subject to treat the signature MP-associated disease or condition. In some embodiments, the IL-1β signature, the Ly6G signature, and the CD66b signature are based on: (1) the presence in the biological sample of the MPs including one or more of IL-1β, Ly6G, and CD66b, respectively; and (2) the number of MPs including one or more of IL-1β, Ly6G, and CD66b, respectively, relative to the total number of MPs in the biological sample. In certain embodiments, the method also includes determining in the biological sample a relative number of the total microparticles that comprise one or more of IL-1β, Ly6G, and CD66b. In some embodiments, the method also includes determining in the biological sample a percentage of the total microparticles that comprise one of more of IL-1β, Ly6G, and CD66b. In some embodiments, the IL-1βsignature is indicated when the percentage of the total number of microparticles in the sample that comprise IL-1βis at least 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. In certain embodiments, the Ly6G signature is indicated when the percentage of the total number of microparticles in the sample that comprise Ly6G is at least 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. In certain embodiments, the CD66b signature is indicated when the percentage of the total number of microparticles in the sample that comprise CD66b is at least 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. In some embodiments, the therapeutic regimen includes administering to the subject confirmed to have the signature MP-associated disease or condition an effective amount of a gelsolin agent to treat the signature MP-associated disease or condition. In some embodiments, admistering the gelsolin agent has a greater therapeutic effect against the signature MP-associated disease or condition in the subject compared to a control therapeutic effect against the signature MP-associated disease or condition. In certain embodiments, the control therapeutic effect is equal to an effect against the signature MP-associated disease or condition in a subject in the absence of administering the gelsolin agent. In some embodiments, the signature MP-associated disease or condition is: hypoxia, decompression sickness, acute hypercarbia, chronic hypercarbia, sleep apnea, steroid-resistant asthma, or hypoxic ischemic encephalopathy, toxic gas toxicity, or asphyxiant gas toxicity. In some embodiments, the toxic gas includes one or both of carbon monoxide and phosgene. In some embodiments, the asphyxiant gas includes one or more of: methane, nitrogen, argon, helium, butane, and propane. In certain embodiments, the signature MP-associated disease or condition is: a retinopathy, Alzheimer's disease, Multiple sclerosis, or a type 2 diabetes sequelae. In certain embodiments, the signature MP-associated disease or condition is one of: chronic obstructive pulmonary disease (COPD), chest wall deformity, a neuromuscular disease, obesity hypoventilation syndrome, respiratory failure, a hypoxia sequelae of a pneumonia, or acute severe asthma. In some embodiments, the neuromuscular disease is myasthenia gravis. In some embodiments, the gelsolin agent includes a gelsolin molecule, a functional fragment thereof, or a functional derivative of the gelsolin molecule. In certain embodiments, the gelsolin molecule is a plasma gelsolin (pGSN). In some embodiments, the gelsolin molecule is a recombinant gelsolin molecule. In some embodiments, the gelsolin agent is administered in a dose from about 3 mg/kg to about 24 mg/kg. In certain embodiments, the administration of the gelsolin agent reduces severity of the signature MP-associated disease or condition in the subject by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100% compared to the severity of the signature MP-associated disease or condition of a control not administered the gelsolin agent. In some embodiments, the method also includes determining a level of severity of the signature MP-associated disease or condition in the subject, wherein a means of the determining includes one or more of: an assay, observing the subject, assessing one or more physiological symptoms of the signature MP-associated disease or condition in the subject, assessing the history of the subject, and assessing a likelihood of survival of the subject. In some embodiments, the physiological symptoms include one or more of: shortness of breath, low blood oxygen saturation, unconsciousness, impaired breathing, headache, vascular permeability, symptoms of poisoning, weakness, cognitive impairment, muscle spasticity, tremor, impaired coordination, visual symptoms, loss of vision, and blindness. In certain embodiments, the history of the subject includes one or more of: exposure to significantly high levels of CO₂, exposure to significantly high levels of CO, scuba diving, and presence at high elevation. In some embodiments, the physiological symptoms include lung pathology. In some embodiments, the administration of the effective amount of the gelsolin agent increases the subject's likelihood of survival compared to a control likelihood of survival. In certain embodiments, the control likelihood of survival is a likelihood of survival in the absence of the administration of the effective amount of the gelsolin agent. In certain embodiments, the likelihood of survival of the subject administered the effective amount of the gelsolin agent is at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, or 100 times higher than the control likelihood of survival. In some embodiments, the administration means of the gelsolin agent is oral, sublingual, buccal, intranasal, intravenous, intramuscular, intrathecal, intraperitoneal, subcutaneous, intradermal, topical, rectal, vaginal, intrasynovial, or intra-ocular administration. In some embodiments, the subject is a mammal, and optionally is a human. In certain embodiments, the biological sample includes and/or is a blood sample. In some embodiments, the signature MP-associated disease or condition is not an infection. In some embodiments, the signature MP-associated disease or condition is a post-infection sequelae. In some embodiments, the subject does not have chronic asthma. In certain embodiments, the subject does not have an active lung infection. In certain embodiments, the gelsolin agent is administered to the subject 1, 2, 3, 4, 5, 6, 7, 8, or more times.

According to another aspect of the invention, a method for treating a signature MP-associated disease or condition in a subject is provided, the method including administering to a subject having or suspected of having the signature MP-associated disease or condition an effective amount of a gelsolin agent wherein the administered gelsolin agent has a greater therapeutic effect against the signature MP-associated disease or condition compared to a control therapeutic effect on the signature MP-associated disease or condition. In some embodiments, the control includes a therapeutic effect of not administering the gelsolin agent. In some embodiments, the therapeutic effect is at least one of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater than the control therapeutic effect. In certain embodiments, the administration of the gelsolin agent reduces severity of the signature MP-associated disease or condition in the subject by at least one of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100% compared to the severity of the signature MP-associated disease or condition of a control not administered the gelsolin agent. In some embodiments, the signature MP-associated disease or condition is: hypoxia, decompression sickness, acute hypercarbia, chronic hypercarbia, sleep apnea, steroid-resistant asthma, hypoxic ischemic encephalopathy, toxic gas toxicity, or asphyxiant gas toxicity. In some embodiments, the toxic gas includes one or more of carbon monoxide and phosgene. In certain embodiments, the asphyxiant gas includes one or more of methane, nitrogen, argon, helium, butane, and propane. In some embodiments, the signature MP-associated disease or condition is: a retinopathy, Alzheimer's disease, Multiple sclerosis, or a type 2 diabetes sequelae. In some embodiments, the signature MP-associated disease or condition is one of: chronic obstructive pulmonary disease (COPD), chest wall deformity, a neuromuscular disease, obesity hypoventilation syndrome, respiratory failure, a hypoxia sequelae of a pneumonia, or acute severe asthma. In some embodiments, the neuromuscular disease is myasthenia gravis. In certain embodiments, the gelsolin agent includes a gelsolin molecule, a functional fragment thereof, or a functional derivative of the gelsolin molecule. In certain embodiments, the gelsolin molecule is a plasma gelsolin (pGSN). In some embodiments, the gelsolin molecule is a recombinant gelsolin molecule. In some embodiments, the gelsolin agent is administered in a dose from about 3 mg/kg to about 24 mg/kg. In some embodiments, the method also includes determining a level of severity of the signature MP-associated disease or condition in the subject, wherein a means of the determining includes one or more of: an assay, observing the subject, assessing one or more physiological symptoms of the signature MP-associated disease or condition in the subject, assessing the history of the subject, and assessing a likelihood of survival of the subject. In certain embodiments, the physiological symptoms include one or more of: shortness of breath, low blood oxygen saturation, unconsciousness, impaired breathing, headache, vascular permeability, symptoms of poisoning, weakness, cognitive impairment, muscle spasticity, tremor, impaired coordination, loss of vision, and blindness. In certain embodiments, the history of the subject includes one or more of exposure to significantly high levels of CO₂, exposure to significantly high levels of CO, and scuba diving, exposure to a toxic gas, exposure to an asphyxiant gas, presence at high elevation, and opioid use. In some embodiments, the assay includes a means for detecting the presence or absence of one or more of an IL-1β signature, a Ly6G signature, and a CD66b signature in a biological sample obtained from the subject. In some embodiments, the IL-1β signature includes the percentage of the total number of microparticles in the sample that comprise IL-1β, the Ly6G signature includes the percentage of the total number of microparticles in the sample that comprise Ly6G, and the CD66b signature includes the percentage of the total number of microparticles in the sample that comprise CD66b. In certain embodiments, the percentage of the total number of microparticles in the biological sample that include IL-1β is at least: 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. In some embodiments, the percentage of the total number of microparticles in the biological sample that include Ly6G is at least: 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. In certain embodiments, the percentage of the total number of microparticles in the biological sample that include CD66b is at least: 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%. In some embodiments, the administration of the effective amount of the gelsolin agent increases the subject's likelihood of survival compared to a control likelihood of survival. In some embodiments, the control likelihood of survival is a likelihood of survival in the absence of the administration of the effective amount of the gelsolin agent. In certain embodiments, the likelihood of survival of the subject administered the effective amount of the gelsolin agent is at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, or 100 times higher than the control likelihood of survival. In certain embodiments, the administration means of the gelsolin agent is selected from: oral, sublingual, buccal, intranasal, intravenous, intramuscular, intrathecal, intraperitoneal, subcutaneous, intradermal, topical, rectal, vaginal, intrasynovial, and intra-ocular administration. In some embodiments, the subject is a mammal. In some embodiments, the subject is a human. In certain embodiments, the subject is a mouse. In certain embodiments, the signature MP-associated disease or condition is not an infection. In some embodiments, the signature MP-associated disease or condition is a post-infection sequelae. In some embodiments, the subject does not have chronic asthma. In some embodiments, the subject does not have an active lung infection. In certain embodiments, the gelsolin agent is administered to the subject 1, 2, 3, 4, 5, 6, 7, 8, or more times.

According to another aspect of the invention, a method for reducing a subject's risk of developing a signature MP-associated disease or condition is provided, the method including: administering to a subject identified as at risk of developing the signature MP-associated disease or condition an effective amount of a gelsolin agent to reduce the subject's risk of developing the signature MP-associated disease or condition. In certain embodiments, administering the gelsolin agent reduces the subject's risk of developing the signature MP-associated disease or condition compared to a control risk of developing the signature MP-associated disease or condition. In some embodiments, the control risk is the risk of developing the signature MP-associated disease or condition in the absence of administering the gelsolin agent. In some embodiments, the subject is identified as at risk of the signature MP disease or condition at least in part on the basis of one of more of: a prior, current, or future activity of the subject; a prior, current, or future potential exposure of the subject; or the presence in the subject of a current disease or condition. In certain embodiments, the prior, current, or future activity of the subject is one or more of: scuba diving, space travel, mining, environmental exploration, and submarine travel. In some embodiments, the prior, current, or future potential exposure of the subject is one or more of exposure to: an asphyxiant gas, a toxic gas, a significantly elevated carbon dioxide (CO₂) level, a significantly elevated carbon monoxide (CO) level, significantly elevated atmospheric pressure, and a non-chronic asthma triggering agent. In certain embodiments, the risk of the subject administered the gelsolin agent of developing the signature MP-associated disease or condition as a result of the a prior, current, or future activity or the a prior, current, or future exposure is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%, lower than the control risk of developing the signature MP-associated disease or condition. In some embodiments, the signature MP-associated disease or condition is: hypoxia, decompression sickness, severe asthma, acute hypercarbia, carbon monoxide (CO) toxicity, toxic gas toxicity, asphyxiant gas toxicity, or carbon dioxide (CO₂) toxicity. In some embodiments, the gelsolin agent includes a gelsolin molecule, a functional fragment thereof, or a functional derivative of the gelsolin molecule. In certain embodiments, the gelsolin molecule is a plasma gelsolin (pGSN). In some embodiments, the gelsolin molecule is a recombinant gelsolin molecule. In some embodiments, the administration of the gelsolin agent increases the subject's likelihood of survival compared to a control likelihood of survival. In certain embodiments, the control likelihood of survival is a likelihood of survival in the absence of the administration of the effective amount of the gelsolin agent. In certain embodiments, the wherein the likelihood of survival of the subject administered the effective amount of the gelsolin agent is at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, or 100 times higher than the control likelihood of survival. In some embodiments, the administration means of the gelsolin agent is selected from: oral, sublingual, buccal, intranasal, intravenous, intramuscular, intrathecal, intraperitoneal, subcutaneous, intradermal, topical, rectal, vaginal, intrasynovial, and intra-ocular administration. In some embodiments, the gelsolin agent is administered to the subject at one or more of prior to, during, and after the activity or potential exposure of the subject. In some embodiments, the gelsolin agent is administered to the subject 1, 2, 3, 4, 5, 6, 7, 8, or more times. In certain embodiments, the subject is a mammal. In certain embodiments, the subject is a human. In some embodiments, the signature MP-associated disease or condition is not an infection. In some embodiments, the signature MP-associated disease or condition is a post-infection sequelae. In certain embodiments, the subject does not have chronic asthma. In certain embodiments, the subject does not have an active lung infection.

According to another aspect of the invention, a method for a prophylactic treatment of an individual (also referred to herein as a subject) susceptible to an occurrence of decompression sickness is provided, the method including: administering to the individual a therapeutically effective amount of a gelsolin agent. In some embodiments, wherein the gelsolin agent includes a gelsolin molecule. In certain embodiments, the gelsolin molecule is a recombinant gelsolin molecule. In some embodiments, gelsolin molecule is administered in a dose from about 3 mg/kg to about 24 mg/kg. In certain embodiments, the gelsolin is administered intravenously. In some embodiments, administering gelsolin inhibits a production of a microparticles of gas in a blood or a tissue of the individual susceptible to an occurrence of decompression sickness.

According to another aspect of the invention, a method for treating decompression sickness in an individual (also referred to herein as a subject) in need of such treatment is provided, the method including: administering to the individual a compound that cleaves filamentous-actin and/or inhibits Interleukin-1β thereby treating the decompression sickness. In some embodiments, the compound is a recombinant gelsolin. In certain embodiments, the compound is an IL-1b inhibitor. In certain embodiments, the compound is canakinumab or

Anakinra. In some embodiments, the compound cleaves filamentous-actin. In some embodiments, the compound is talin, cofilin, twinfilin, adseverin, ECP32/grimelysin or protealysin. In certain embodiments, the method also includes administering to the individual two or more compounds that cleave filamentous-actin and/or inhibits Interleukin-1β in amounts effective to treat the decompression sickness.

### BRIEF DESCRIPTION OF THE DRAWINGS

These and other features, aspects, and advantages of the embodiments of the present disclosure is better understood when the following detailed description is read with reference to the accompanying drawings in which like characters represent like parts throughout the drawing, wherein:

FIG. 1 provides graphs illustrating results showing changes in blood from human research subjects. The concentrations of pGSN and IL-1β were measured in plasma samples and blood-borne MPs were quantified pre-, at- and post-exposure to 300 kPa as described in Methods. Individual data points are shown and below each plot are mean+SE, n=6 for each sample, * indicates significantly different from pre-exposure, p<0.05, RM ANOVA.

FIG. 2 shows bar graphs illustrating results of changes in experimental mice. Male mice were exposed to air at ambient pressure (control) or for 2 hours to 790 kPa air, decompressed and euthanized 2 hours later (Deco). Where indicated air-exposed control mice were injected intravenously with 27 mg/kg rhu-pGSN (Control+pGSN) and euthanized 4 hours later. Other mice were injected with rhu-pGSN prior to pressurization (pGSN+Deco) or immediately after decompression (Deco+pGSN), and others injected intravenously with the carrier buffer used to suspend rhu-pGSN (Vehicle+Deco), and these groups euthanized 2 hours after decompression. The concentrations of pGSN and IL-1β were measured in plasma samples by mouse-specific ELISAs and blood-borne MPs were quantified as described in Methods. Data are mean+SE, the (n) for each sample is shown, * indicates significantly different from control, p<0.05, ANOVA.

FIG. 3 is a Western blot illustrating biotinylation of microparticle (MP) proteins. MPs from control and decompressed male mice were isolated, incubated with 200 μg/ml rhu-pGSN (shown as +pGSN) or just PBS, and then biotinylated as described in Methods section of Examples herein. MPs were then lysed in SDS buffer and protein from 45,500 MPs loaded into each lane for SDS-PAGE. Western blots probed for biotin and for β-actin are shown. Probing for IL-1β did not demonstrate bands (not shown). Molecular weight standards (in kDa) are shown at left.

FIG. 4 is a Western blot illustrating biotinylated versus non-biotinylated MPs separation. MPs from control and decompressed male mice were isolated, biotinylated and then lysed. Samples were incubated with magnetic streptavidin beads as described in Methods section in Examples herein and passed through a magnet to separate biotinylated (shown as +Biotin) from non-biotinylated proteins (shown as −Biotin). Protein from 165,000 MPs was loaded into each lane for SDS-PAGE. Western blots probed for β-actin and biotin are shown. Probing for IL-1β did not demonstrate bands (not shown). Molecular weight standards (in kDa) are shown at left.

FIG. 5A-C provides two graphs and a table showing effect of rhu-pGSN on MPs from control and decompressed mice. Blood was obtained from control or decompressed male mice and centrifuged as described in Methods. MPs suspensions were divided and where shown at time 0, 200 μg/ml rhu-pGSN was added. At 30 minute intervals samples were fixed. The number of remaining MPs are shown in FIG. 5A. FIG. 5B shows the % of MPs that bind anti-gelsolin antibody and phalloidin. Values in bold are statistically significantly different from the values as time 0 (p<0.05, ANOVA). FIG. 5C show the % of particles that bound fluorescent phalloidin. Data are mean+SE, n=5 for each sample, * indicates significantly different from the value at time 0, p<0.05, RM-ANOVA.

FIG. 6 shows five graphs illustrating the effect of rhu-pGSN on human neutrophils and MPs. Neutrophils were isolated, incubated for 30 minutes in ambient air or at 790 kPa and decompressed. At time 0, rhu-pGSN (200 μg/ml) was added and at 30-minute intervals portions of samples were fixed and processed as described in Methods to quantify MPs, binding of anti-gelsolin antibody and fluorescent phalloidin. Data are mean+SE, n=4 for each sample, * indicates significantly different from the value at time 0, p<0.05, RM-ANOVA.

### BRIEF DESCRIPTION OF SEQUENCES

SEQ ID NO: 1 is an amino acid sequence of human plasma gelsolin having GenBank® Accession No. X04412:

MAPHRPAPALLCALSLALCALSLPVRAATASRGASQAGAPQ
GRVPEARPNSMVVEHPEFLKAGKEPGLQIWRVEKFDLVPV
PTNLYGDFFTGDAYVILKTVQLRNGNLQYDLHYWLGNECS
QDESGAAAIFTVQLDDYLNGRAVQHREVQGFESATFLGYF
KSGLKYKKGGVASGFKHVVPNEVVVQRLFQVKGRRVVRAT
EVPVSWESENNGDCFILDLGNNIHQWCGSNSNRYERLKAT
QVSKGIRDNERSGRARVHVSEEGTEPEAMLQVLGPKPALP
AGTEDTAKEDAANRKLAKLYKVSNGAGTMSVSLVADENPF
AQGALKSEDCFILDHGKDGKIFVWKGKQANTEERKAALKT
ASDFITKMDYPKQTQVSVLPEGGETPLFKQFFKNWRDPDQ
TDGLGLSYLSSHIANVERVPFDAATLHTSTAMAAQHGMDD
DGTGQKQIWRIEGSNKVPVDPATYGQFYGGDSYIILYNYR
HGGRQGQIIYNWQGAQSTQDEVAASAILTAQLDEELGGTP
VQSRVVQGKEPAHLMSLFGGKPMIIYKGGTSREGGQTAPA
STRLFQVRANSAGATRAVEVLPKAGALNSNDAFVLKTPSA
AYLWVGTGASEAEKTGAQELLRVLRAQPVQVAEGSEPDGF
WEALGGKAAYRTSPRLKDKKMDAHPPRLFACSNKIGRFVI
EEVPGELMQEDLATDDVMLLDTWDQVFVWVGKDSQEEEKT
EALTSAKRYIETDPANRDRRTPITVVKQGFEPPSFVGWFL
GWDDDYWSVDPLDRAMAELAA.

### DETAILED DESCRIPTION

The present invention is based, in part, on the discovery that the presence of specific microparticle (MP) “signatures” can be used to detect the presence or absence of MP-associated diseases and disorders in subjects. It has now been discovered that MPs comprising at least one of IL-1β, lymphocyte antigen 6 complex locus G6D (Ly6G) (mouse), or CD66b (human) can be detected and used to identify the presence of a signature MP-associated disease or condition in a subject. Certain embodiments of methods of the invention can be used to identify a subject as having a MP-associated disease or condition, or to be at risk of having a microparticle-associated disease or condition. It is now possible to use embodiments of methods of the invention to identify a subject in need of a treatment for a MP-associated disease or condition and to select a treatment regimen for the subject based on the identification of the disease or condition. In some embodiments a selected treatment regimen can be administered in an amount effective to treat the MP-associated disease or condition in the subject. Certain methods of the invention include a treatment regimen comprising administering to a subject identified as having or at risk of a MP-associated disease or condition, a therapeutic composition comprising a gelsolin agent.

Certain embodiments of methods of the invention include detecting in a biological sample from a subject a MP signature, such as an IL-1β MP signature, an LY6G MP signature, and a CD66b MP signature, which are indicated based on the presence and number (relative to the total MP number) of MPs in the biological sample that comprise at least one of IL-1β, LY6G, and CD66b, respectively. In some embodiments of methods of the invention, identification of one or more of an IL-1β signature, an LY6G signature, and a CD66b signature, as described herein can be used to (1) confirm whether or not a subject has an MP-associated disease or condition; (2) select a therapeutic regimen with which to treat the subject confirmed as having the MP-associated disease or condition; and (3) administering the selected therapeutic regimen to the subject.

It has now been determined that plasma gelsolin (pGSN) levels are decreased in subjects in response to stress imposed by high pressure and subsequent decompression, and that administering gelsolin to the subjects ameliorates injuries in decompression sickness and other signature MP-associated diseases and conditions. Certain embodiments of methods of the invention can be used to prevent and/or treat a subject by administering a gelsolin agent to the subject in an amount effective to reduce, prevent and/or reduce the severity of a signature MP-associated disease or condition. Certain methods of the invention include administering a gelsolin agent to a subject with a signature MP-associated disease or condition, or administering a gelsolin agent prophylactically to a subject at risk of an MP-associated disease or condition.

### Certain Definitions

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, the term “contacting” refers to any suitable method of bringing an inhibitor, a compound or a pharmaceutical composition into contact with a cell. For in vivo applications, any known method of administration is suitable as described herein.

The terms pGLN and pGSN are used herein interchangeably.

### Microparticles

Microparticles (MPs), which are also known as microvesicles and extracellular vesicles, are cell-derived structures, which generally range from 50 nm to 1,000 nm in diameter. Naturally occurring MPs are heterogeneous and may serve as communication means between cells and tissues. MPs are formed from cells by a process of budding from the cell's plasma membrane and MPs released from a cell may comprise molecules such as nucleic acids, proteins, and lipids that are specific to their cell origin [see for example van Niel, G. et al., (2018) Nature Reviews. Vol. 19:213-228].

It has now been determined that the presence of specific MP signatures can be used to identify a physiological status of a subject as having an MP-associated disease or condition. The term “signature” as used herein in reference to MPs means one or more characteristics of the MP. Characteristics that may determine an MP's signature include but are not limited to: whether an MP comprises certain components and the number or amount of such MPs. The term “component” used herein in reference to an MP signature means a molecule that is part of the MP's membrane and/or a molecule internal to the MP's membrane. For example, though not intended to be limiting a component of an MP may be a surface protein of the MP. As another non-limiting example, a component of an MP may be a protein or nucleic acid molecule that is internal to the MP. Non-limiting examples of surface proteins that can be detected using methods of the invention, and are components of certain MPs are IL-1β, Ly6G, and CD66b,

In addition to identifying specific components of MPs, methods of the invention may also include determining an amount or number of MPs comprising a particular component of interest. In certain embodiments of methods of the invention, an amount or number of MPs comprising the particular component of interest is determined relative to the number of MPs that do not comprise the component of interest. For example, certain embodiments of methods of the invention include detecting MPs in a biological sample. The terms “detecting” or “detection” as used herein in relation to determining the presence of a signature MP-associated disease or condition include identifying the presence in the biological sample of MPs comprising one or more specific components of interest and/or determining a number or amount of the identified MPs relative to the total number of MPs in the biological sample. Detecting MPs comprising specific components of interest and/or determining the relative abundance of the MPs comprising the specific components of interest indicates an MP signature in the biological sample that can be used to confirm the presence of a signature MP-associated disease or condition in a subject from whom the biological sample was obtained.

An example of an MP component that can be detected and utilized to confirm the presence in a subject of a signature MP-associated disease or condition with a method of the invention is interleukin-1B (IL-1β), which is also known in the art as leukocytic pyrogen, leukocytic endogenous mediator, mononuclear cell factor, and lymphocyte activating factor. Another example of an MP component that can be detected and utilized to confirm the presence in a subject of a signature MP-associated disease or condition with a method of the invention is lymphocyte antigen 6 complex locus protein G6D (Ly6D), which is also known in the art at least as megakaryocyte-enhanced gene transcript 1 protein, G6D, NG25, LY6-D, MEGT1, and C6orf23. An LY6D signature can be used to identify a mouse signature MP-associated disease or condition. Another example of an MP component that can be detected and utilized to confirm the presence in a subject of a signature MP-associated disease or condition with a method of the invention is CD66b, which is also known in the art at least as CD67, CGM6, and NCA-95. A CD66b signature can be used to identify a mouse signature MP-associated disease or condition Certain embodiments of methods of the invention include detecting MPs with a signature of one or more of IL-1β, Ly6G, and CD66b in a biological sample obtained from a subject, wherein detecting the signature confirms the presence of a signature MP-associated disease or condition in the subject.

Some embodiments of methods of the invention include detecting the presence of one or a plurality of MPs in a biological sample, identifying in the detected MP(s) the presence or absence of MPs comprising one or more of IL-1β, Ly6G, and CD66b, and optionally determining an amount of MPs detected as comprising IL-1β, Ly6G, and/or CD66b in the biological sample. The determined amount may be measured as a number of the MPs comprising one or more of IL-1β, Ly6G, and CD66b, relative to a total amount of MPs detected in the biological sample. A relative amount of an MP comprising one or more of IL-1β, Ly6G, and CD66b in a biological sample may be expressed as a proportion of the total MPs in the biological sample (for example as a ratio) and/or as a percentage of the total MPs in the biological sample. It has been determined that the proportion and/or percentage of the MPs in a biological sample that comprise one or more of IL-1β, Ly6G, and CD66b corresponds to the presence or absence of a signature MP-associated disease or condition in the subject from whom the biological sample was obtained. It will be understood a biological sample can be tested for the presence of each of IL-1β, Ly6G, and CD66b independent of the other two components. Thus, some embodiments of methods of the invention, include detecting MPs comprising IL-1β and identifying the presence and/or relative number of MPs comprising IL-1β to determine whether the biological sample has an MP IL-1β signature. Some embodiments of methods of the invention, include detecting MPs comprising LY6G and identifying the presence and/or relative number of MPs comprising LY6G to determine whether the biological sample has an LY6G signature. Certain embodiments of methods of the invention, include detecting MPs comprising CD66b and identifying the presence and/or relative number of MPs comprising CD66b to determine whether the biological sample has a CD66b signature. In some embodiments of the invention a biological sample is obtained from a human subject and the detection of either one or both of an IL-1β MP signature and a CD66b MP signature is determined in the biological sample, which indicates the presence of a signature MP-associated disease or disorder in the subject. In certain embodiments of the invention a biological sample is obtained from a mouse, or other rodent and either one or both of an IL-1β MP signature and an Ly6G MP signature is determined in a biological sample, which indicates the presence of a signature MP-associated disease or disorder in the subject.

Some embodiments of the invention include detecting in a biological sample obtained from a subject a percentage of the total number of MPs that are MPs comprising one or more of IL-1β, Ly6G, and CD66b. In some embodiments, an IL-1β signature comprises the percentage of the total number of MPs in a biological sample that are MPs comprising IL-1β. In some embodiments, a Ly6G signature comprises the percentage of the total number of MPs in a biological sample that are MPs comprising Ly6G. In some embodiments, a CD66b signature comprises the percentage of the total number of MPs in a biological sample that are MPs comprising CD66b. In some embodiments of methods of the invention, an IL-1βsignature, a Ly6G signature, or a CD66b signature identified in a biological sample obtained from a subject is a percentage of the total MPs that are MPs comprising IL-1β, Ly6G, or CD66b, respectively, that is least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42, %, 43%, 44%, 45%, 46%, 47%, 48%, 49%, 50%, 51%, 52%, 53%, 54%, 55%, 56%, 57%, 58%, 59%, 60%, 61%, 62, %, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the total MPs in the biological sample, which confirms the presence of a signature MP-associated disease or condition in the subject. It will be understood that IL-1β, Ly6G, and CD66b signatures may be expressed as ratios that indicate an amount or number of MPs comprising one or more of IL-1β, Ly6G, and CD66b, respectively, in a biological sample relative to a total amount or number of MPs in the biological sample, and that the ratios may be used to identify subjects having a signature MP-associated disease or condition as described herein.

### Treatment Selection

As described herein, a therapeutic regimen may be selected for a subject based at least in part on the detecting in a biological sample obtained from the subject, the presence of MPs comprising one or more of IL-1β, Ly6G, and CD66b, and/or determining a relative amount of MPs in the biological sample that comprise the one or more of IL-1β, Ly6G, and CD66b versus MPs that do not comprise the one or more of IL-1β, Ly6G, and CD66b, respectively. In some embodiments of the invention, a treatment regimen selection may also be based, at least in part, on the severity of the signature MP-associated disease or condition in the subject. In some embodiments of the invention, a selected treatment regimen may include administering to the subject an effective amount of a gelsolin agent to treat the signature MP-associated disease or condition, as well as one or more additional treatments appropriate for the specific disease or condition in the subject. As a non-limiting example, an additional treatment for a subject identified as having or being at risk of having decompression sickness may be one or more of: positioning the subject in a hyperbaric chamber, hyperbaric treatment, surgery, thrombolytic therapy, anticoagulant therapy, and administration of supplemental oxygen, etc. It will be understood that in the case of identifying a subject as having or being at risk of a different signature MP-associated disease or and condition the selected treatment regimen comprising administration of a gelsolin agent to the subject may also include one or more additional treatments appropriate for the particular signature MP-associated disease or condition. Upon a determination of the presence or a risk of a signature MP-associated disease or condition in a subject, a practitioner will, without undue experimentation, be aware of and able to select one or more treatments for inclusion, in addition to the administration of a gelsolin agent, in a therapeutic regimen for the subject.

### Gelsolin Agents

Gelsolin is a highly conserved, multifunctional protein, initially described in the cytosol of macrophages and subsequently identified in many vertebrate cells [see for example Piktel E. et al., Int J Mol Sci 2018; 19:E2516; Silacci P. et al., Cell Mol Life Sci 2004; 61:2614-23.) A unique property of gelsolin is that its gene expresses a splice variant coding for a distinct plasma isoform (pGSN), which is secreted into extracellular fluids and differs from its cytoplasmic counterpart (cGSN) by expressing an additional sequence of 25 amino acids. pGSN normally circulates in mammalian blood at concentrations of 200-300 μg/ml, placing it among the most abundant plasma proteins. The term “gelsolin agent” as used. herein means a composition that includes a gelsolin molecule. In some embodiments of methods of the invention, a gelsolin molecule may be a functional fragment or functional derivative of a full-length, natural, parent gelsolin molecule. In some embodiments of the invention, a gelsolin agent only includes one or more of the gelsolin molecule, a functional fragment thereof, or a functional derivative of the gelsolin molecule. In certain embodiments of the invention a gelsolin agent may include one of more additional components, non-limiting examples of which are detectable labels, carriers, delivery agents, etc. In certain aspects of the invention a gelsolin molecule is a plasma gelsolin (pGSN) and in certain instances, a gelsolin molecule is a cytoplasmic GSN. A gelsolin molecule included in compositions and methods of the invention may be a recombinant gelsolin molecule.

As used herein, the term “gelsolin agent” is a compound that includes an exogenous gelsolin molecule. The term “exogenous” as used herein in reference to a gelsolin molecule means a gelsolin molecule administered to a subject, even if the same gelsolin molecule is naturally present in the subject, which may be referred to as an endogenous gelsolin molecule. A gelsolin agent included in a method of the invention may be a wild-type gelsolin molecule (such as GenBank accession No.: X04412, the amino acid sequence of which is set forth herein as SEQ ID NO: 1), an isoform, an analog, a functional variant, a functional fragment, or afunctional derivative of a gelsolin molecule. It will be understood that in some embodiments of the invention an administered gelsolin molecule is a gelsolin polypeptide and in certain embodiments of methods of the invention an administered gelsolin molecule is a gelsolin polypeptide-encoding polynucleotide.

Some embodiments of methods of the invention include administration of a “gelsolin analog,” which as used herein refers to a compound substantially similar in function to either the native gelsolin or to a fragment thereof. Gelsolin analogs include biologically active amino acid sequences substantially similar to the gelsolin sequences and may have substituted, deleted, elongated, replaced, or otherwise modified sequences that possess bioactivity substantially similar to that of gelsolin. For example, an analog of gelsolin is one that does not have the same amino acid sequence as gelsolin but that is sufficiently homologous to gelsolin so as to retain the bioactivity of gelsolin. Bioactivity can be determined, for example, by determining the properties of the gelsolin analog and/or by determining the ability of the gelsolin analog to reduce or prevent the effects of a signature MP-associated disease or condition. Gelsolin bioactivity assays known to those of ordinary skill in the art.

Certain embodiments of methods of the invention include fragments of a gelsolin molecule. The term “fragment” is meant to include any portion of a gelsolin molecule that provides a segment of gelsolin that maintains at least a portion or substantially all of a level of bioactivity of the “parent” gelsolin. The term gelsolin fragment is meant to include gelsolin fragments made from any source, such as, for example, from naturally-occurring peptide sequences, synthetic or chemically-synthesized peptide sequences, and genetically engineered peptide sequences. The term “parent” as used herein in reference to a gelsolin fragment or derivative molecule means the gelsolin molecule from which the sequence of the fragment or derivative originated.

In certain embodiments of methods of the invention, a gelsolin fragment is a functional fragment and retains at least some up to all of the function of its parent gelsolin molecule. Methods of the invention, may in some embodiments include administration of a “variant” of gelsolin. As used herein a gelsolin variant may be a compound substantially similar in structure and bioactivity either to native gelsolin, or to a fragment thereof. In certain aspects of the invention, a gelsolin variant is referred to as a functional variant, and retains at least some up to all of the function of its parent gelsolin molecule.

Gelsolin derivatives are also contemplated for inclusion in embodiments of methods of the invention. A “functional derivative” of gelsolin is a derivative that possesses a bioactivity that is substantially similar to the bioactivity of gelsolin. By “substantially similar” is meant activity which may be quantitatively different but qualitatively the same. For example, a functional derivative of gelsolin could contain the same amino acid backbone as gelsolin but also contains other modifications such as post-translational modifications such as, for example, bound phospholipids, or covalently linked carbohydrate, depending on the necessity of such modifications for the performance of a therapeutic method of the invention. As used herein, the term is also meant to include a chemical derivative of gelsolin. Such derivatives may improve gelsolin's solubility, absorption, biological half-life, etc. The derivatives may also decrease the toxicity of gelsolin, or eliminate or attenuate any undesirable side effect of gelsolin, etc. Derivatives and specifically, chemical moieties capable of mediating such effects are disclosed in Remington, The Science and Practice of Pharmacy, 2012, Editor: Allen, Loyd V., Jr, 22^{nd} Edition). Procedures for coupling such moieties to a molecule such as gelsolin are well known in the art. The term “functional derivative” is intended to include the “fragments,” “variants,” “analogues,” or “chemical derivatives” of gelsolin.

### Signature MP-Associated Diseases and Conditions

Methods of the invention can be used to identify and/or treat a signature MP-associated disease or condition in a subject. The term “signature MP-associated diseases and conditions” as used herein encompasses diseases and conditions in which MPs comprising one or more of: IL-1β, Ly6G, and CD66b are produced in an amount higher than would be produced in the absence of the signature MP-associated disease or condition, and the presence and/or amount of such MPs can be used to determine the presence of the disease or condition in a subject.

In some embodiments of methods of the invention, a signature MP-associated disease or condition is a disease or condition in which there is a physiological reduction in availability and/or access to oxygen by tissues in a subject. A non-limiting example of such a signature MP-associated disease or condition is decompression sickness, which is also known as DCS, divers' disease, the bends, aerobullosis, and caisson disease. In this condition, depressurization of a subject, for example when ascending from a deep dive, ascending to high elevation, results in gases dissolved in body tissues of a subject coming out of solution. Resulting symptoms may include joint pain, skeletal pain, breathing difficulty, paralysis, unconsciousness, weakness, headache, neurological disturbances, etc. Less severe episodes of DCS may include symptoms that involve the skin, muscles, and lymphatic systems and episodes of more severe DCS may additionally include symptoms indicating damage in the subject's nervous system and other organs.

Non-limiting examples of other signature MP-associated diseases and conditions that can be identified in a subject using an embodiment of a method of the invention and treated by administration of a gelsolin agent to the subject are: hypoxia, decompression sickness, acute hypercarbia, chronic hypercarbia, sleep apnea, steroid-resistant asthma, hypoxic ischemic encephalopathy, chronic obstructive pulmonary disease (COPD), chest wall deformity, a neuromuscular disease, (such as but not limited to myasthenia gravis), obesity hypoventilation syndrome, respiratory failure, a hypoxia sequelae of a pneumonia, acute severe asthma, and opioid overdose.

Additional signature MP-associated diseases or conditions that can be identified in a subject using a method of the invention and treated by administration of a gelsolin agent to the subject are toxic gas toxicity and asphyxiant gas toxicity. Non-limiting examples of toxic gases are: carbon monoxide, elevated levels of carbon dioxide, and phosgene gas. An asphyxiant gas is a non-toxic or minimally toxic gas that reduces or replaces normal oxygen concentration in air that is breathed. Non-limiting examples of asphyxiant gases are: methane, nitrogen, argon, helium, butane, and propane. It will be understood that a subject exposed to toxic gases or asphyxiant gases does not always develop a signature MP-associated disease or condition and whether an exposure of the subject to one or more of a toxic or asphyxiant gas results in a signature MP-associated disease or condition depends in part on factors such as the length of the exposure, the level of exposure, the concentration of the toxic or asphyxiant gas encountered by the subject, etc. The term “significantly high levels” as used herein in reference to toxic gas toxicity and asphyxiant gas toxicity means amounts, levels and/or concentrations of a gas sufficiently high to result in a signature MP-associated disease or disorder in the subject.

Additional non-limiting examples of signature MP-associated diseases and conditions that can be identified in a subject using an embodiment of a method of the invention and treated by administration of a gelsolin agent to the subject are: Type 2 diabetes sequelae such as but not limited to: vascular damage, vascular leakage, diabetic retinopathy (DR); auto-inflammatory diseases such as but not limited to: Cryopyrin-associated Periodic Syndrome (CAPS), crystal-induced arthritis, neutrophilic asthma; neuro-inflammatory disease such as but not limited to: Alzheimer's disease, Multiple Sclerosis, Lewy body dementia; age-related macular degeneration (AMD), dry eye, Keratoconjunctivitis sicca (KCA), ischemic retinopathy. Retinopathy, retinopathy of prematurity (ROP), blindness, loss of vision; Retinal hypoxia-associated diseases such as but not limited to: retinal ganglion cell (RGC) death, central retinal artery occlusion, ischemic central retinal vein thrombosis, complications of diabetic eye disease sequelae, and glaucoma.

Diseases and conditions referred to herein as signature MP-associated diseases or conditions are diseases and conditions in which MPs comprising one or more of: IL-1β, Ly6G, and CD66b are produced in an amount higher than would be produced in the absence of the signature MP-associated disease or condition. As described herein, a subject can be determined to have a signature MP-associated disease or conditions by a method of detecting in a biological sample obtained from the subject, the presence of signature MPs comprising one or more of: IL-1β, Ly6G, and CD66b. Following determination of an IL-1β MP signature, an Ly6G MP signature, and/or a CD66b MP signature in a sample from the subject, methods of the invention may include selecting a therapeutic regimen for the subject, wherein the therapeutic regimen comprises administering a gelsolin agent to the subject. A therapeutic regimen of the invention may also include one or more additional therapeutic actions or administered medicaments, depending on the specific signature MP-associated disease or condition, the severity of the signature MP-associated disease or condition, or other factors of which a practitioner will be aware as factors for consideration in selecting a treatment. Methods of the invention may also include administering a selected therapeutic regimen to the subject.

It will be understood that the signature MP-associated diseases and conditions set forth herein are not infections, although in some embodiments of methods of the invention, a signature MP-associated disease or condition may be a post-infection sequelae. In certain embodiments of the invention the subject does not have an active lung infection. A signature MP-associated condition may be an asthmatic condition that is distinct from chronic asthma in that it may be caused by an inhaled gas or other substance. In some embodiments of the invention, a subject does not have chronic asthma. In some embodiments of methods of the invention, the MP-associated disease or condition determined to be present in the subject is not associated with or resulting from an active infection in the subject.

### Risk Reduction

The invention, in part, includes methods of reducing a subject's risk of developing a signature MP-associated disease or condition. Certain embodiments of risk reduction methods of the invention include administering a gelsolin agent to a subject identified as at risk of developing a signature MP-associated disease or condition, and the gelsolin agent is administered in an amount effective to reduce the subject's risk of developing the signature MP-associated disease or condition. Efficacy of a method of the invention to reduce a subject's risk may be determined by comparing results of administering a gelsolin agent to a subject with control results. In some embodiments of the invention, a gelsolin agent administered to a subject reduces the subject's risk of developing a signature MP-associated disease or condition compared to a control risk of developing the signature MP-associated disease or condition, wherein the control risk is a risk of a subject in essentially identical circumstances developing the signature MP-associated disease or condition in the absence of administering the gelsolin agent.

Certain embodiments of methods of the invention include identifying a subject as at risk of developing a signature MP disease or condition, and the identification may be based, at least in part, on factors such as but not limited to: a prior, current, or future activity of the subject and a prior, current, or future potential exposure of the subject to an agent or element that is believed may cause the signature MP-associated disease or condition to develop in the subject. The term “activity” used herein in reference to risk of a signature MP-associated disease or condition encompasses behaviors that increase a subject's risk of the signature MP-associated disease or condition. Non-limiting examples of prior, current, and future activities of a subject are: scuba diving, caving, mountain climbing, high elevation travel, space travel, extravehicular activities in space, deep mining, environmental exploration, and submarine travel. As used herein the term environmental exploration means a subject is present in or has sufficient proximity to a physical environment in which there is a risk to the subject of exposure to an agent or element that can result in a reduced-oxygenation-associated disease or disorder in the subject. Non-limiting examples of such environments are: volcanos, fires, industrial accidents, high altitude locations, deep underwater locations, etc. Non-limiting examples of agents or elements to which a subject may be exposed in a prior, current, or future event or activity that may be believed to result in a signature MP-associated disease or condition are a toxic gas, an asphyxiant gas, a significantly elevated carbon dioxide (CO₂) level, a significantly elevated carbon monoxide (CO) level, significantly elevated atmospheric pressure, and a non-chronic asthma triggering agent.

In addition to activities or future activities that may indicate a risk of a subject developing a signature MP-associated disease or condition, the presence of an existing disease or condition in a subject may indicate a risk of the subject developing a signature MP-associated disease or condition. For example, though not intended to be limiting, a subject might have type 2 diabetes and thus be considered to be at risk for a signature MP-associated disease or condition such as type 2 diabetes sequelae, diabetic retinopathy, etc.

Certain embodiments of methods of the invention include administering a gelsolin agent to a subject identified as at risk of developing a signature MP-associated disease or condition. In some embodiments of therapeutic methods of the invention a gelsolin agent includes a gelsolin molecule, a functional fragment of a gelsolin molecule, or a functional derivative of a gelsolin molecule. In some embodiments of the invention, the administered gelsolin agent comprises a plasma gelsolin (pGSN). An administered gelsolin agent may, in some embodiments of the invention, comprise a recombinant gelsolin molecule.

Certain embodiments of methods of the invention include administering the gelsolin agent to a subject in an amount effective to reduce the subject's risk of developing a signature MP-associated disease or condition, and/or to reduce the severity of a signature MP-associated disease or condition present in a subject. In some embodiments, a therapeutically effective amount refers to that amount of the inhibitor and/or compound being administered to a subject that is sufficient to prevent progression of a disease or condition, such as a signature MP-associated disease or condition. Administration of the gelsolin agent may reduce the risk of a subject developing the signature MP-associated disease or condition as a result of the a prior, current, or future activity or a prior, current, or future exposure by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42,%, 43%, 44%, 45%, 46%, 47%, 48%, 49%, 50%, 51%, 52%, 53%, 54%, 55%, 56%, 57%, 58%, 59%, 60%, 61%, 62,%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, compared to the percent control risk of the subject developing the signature MP-associated disease or condition. For example, if a subject's risk of developing a signature MP-associated disease or condition in an upcoming deep scuba diving activity is 20%, administering an effective amount of a gelsolin agent to the subject may reduce the subject's risk of the 20% risk down to less than 19%, 18%, 17%, 16%, 15%, 14%, 13%, 12%, 11%, 10%, 9%, 8%, 7%, 6%, 5%, 4%, 3%, 2%, 1% risk, or to 0% risk.

In certain embodiments of methods of the invention, administering an effective amount of a gelsolin agent to a subject based on a prior, current, or future activity of the subject and/or a prior, current, or future potential exposure of the subject to an agent or element that causes the signature MP-associated disease or condition to develop in a subject, increases the subject's likelihood of survival compared to a control likelihood of survival. In some instances a control likelihood of survival is a likelihood of survival of subject in an essentially identical activity or exposure in the absence of the administration of the effective amount of the gelsolin agent. Administration of an effective amount of the gelsolin agent to a subject in need of such treatment can increase the likelihood of survival of the subject to least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, or 100 times higher than the control likelihood of survival. Another way of expressing a change in likelihood of survival is in reduction in the percent likelihood of death. For example, as a result of treatment with a gelsolin agent, a subject may have a risk of death that is up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42,%, 43%, 44%, 45%, 46%, 47%, 48%, 49%, 50%, 51%, 52%, 53%, 54%, 55%, 56%, 57%, 58%, 59%, 60%, 61%, 62,%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of the risk of death of a control subject not treated with the gelsolin agent. For example, if a control risk of death from a signature MP-associated disease or condition is 20%, administering an effective amount of a gelsolin agent to a subject identified as having the signature MP-associated disease of condition may have a risk of death resulting from the MP-associated disease or condition that is reduced to less than 20%, 19%, 18%, 17%, 16%, 15%, 14%, 13%, 12%, 11%, 10%, 9%, 8%, 7%, 6%, 5%, 4%, 3%, 2%, 1% risk, or the risk of the subject's death due to the signature MP-associated disease or condition is reduced to 0%.

The timing of administration of a gelsolin agent may be determined based on the timing of the activity and/or potential exposure of the subject. In some embodiments, the gelsolin agent is administered to the subject at one or more of prior to, during, and after the activity or potential exposure of the subject. The gelsolin agent may be administered to a subject determined to be in need such treatment once, or multiple times. Multiple administrations of a gelsolin agent means the gelsolin agent is administered to a subject 2, 3, 4, 5, 6, 7, 8, 9, 10, or more times. It will be understood that administration of a gelsolin agent may be done in combination with additional treatments for a signature MP-associated diseases or condition, non-limiting examples of which are: oxygen administration, intubation, hyperbaric treatment, etc.

Some embodiments of the invention include a method of treating decompression sickness in an individual in need of such treatment, comprising the steps of administering to the individual a compound that cleaves filamentous-actin and/or inhibits Interleukin-1β thereby treating the decompression sickness. In one aspect, the compound is a recombinant gelsolin or an analogue thereof. In another aspect, the compound is an IL-1b inhibitor. Representative examples of IL-1b inhibitors include but are not limited to canakinumab, and the IL-1b receptor inhibitor Anakinra. In another aspect, the compound is a compound that cleaves filamentous-actin. Representative examples of a compound that cleaves filamentous-actin include but are not limited to talin, cofilin, twinfilin, adseverin, and the bacterial proteases ECP32/grimelysin and protealysin. In some embodiments of methods of the invention a gelsolin agent is administered in combination with agent that cleaves filamentous-actin and/or inhibits Interleukin-1β IL-1b inhibitor as a treatment of a MP-associated disease or condition, such as but not limited to decompression sickness.

### Therapeutic Compositions and Methods and Monitoring Efficacy

Methods of the invention include producing a therapeutic effect in a subject that has a signature MP-associated disease or condition to reduce and treat the signature MP-associated disease or condition. The term “therapeutic effect” as used herein in reference to an agent such as a gelsolin agent means a therapeutic effect of the gelsolin agent when it is administered to a subject having a signature MP-associated disease or condition. A therapeutic effect of gelsolin (also referred to herein as a “response” to a treatment method of the invention) can be determined, for example, by detecting one or more physiological effects of the treatment, such as the decrease or lack of symptoms following administration of the treatment. Additional means of monitoring and assessing a signature MP-associated disease or condition in a subject, and ways to assess and determine one or more of a level, severity, change in severity, etc. of a signature MP-associated disease or condition in subject are known in the art and can be used to assess the signature MP-associated condition in a subject following a treatment comprising administering a gelsolin agent to a subject. Non-limiting examples of physiological symptoms that can be assessed in certain embodiments of methods of the invention are provided elsewhere herein and will be known in the art and routinely assessed for specific diseases and conditions.

Some embodiments of a method of the invention may also comprise determining efficacy of an administered therapeutic regimen. For example, an amount of MPs comprising one of more of IL-1β, Ly6G, and CD66b can be determined in a first biological sample obtained from a subject that has a signature MP-associated disease or condition and the amount of MPs comprising the one or more of IL-1β, Ly6G, and CD66b in a biological sample obtained from the subject at a subsequent time can be determined and the results of the determinations compared. If the detected amount of IL-1β, Ly6G, and/or CD66b in the initial sample is higher than the detected amount in the subsequent sample it may indicate decrease in severity of the signature MP-associated disease or condition in the subject. If the amount MPs comprising one or more of IL-1β, Ly6G, and/or CD66b in an initially obtained biological sample is lower than the amount of MPs comprising one or more of IL-1β, Ly6G, and/or CD66b detected in subsequently biological sample obtained from the subject, it may indicate the onset of, and/or an increase in the severity of the signature MP-associated disease or condition in the subject.

In instances where a subject is administered a gelsolin agent after the time a first biological sample is obtained from the subject a subsequent biological sample may be obtained after the administration and a difference in an amount of MPs comprising IL-1β, Ly6G, and/or CD66b in the first biological sample and the subsequent biological sample may indicate a level of efficacy of the administered gelsolin to treat the signature MP-associated disease or condition in the subject. If a detected amount of MPs comprising IL-1β, Ly6G, and/or CD66b in a biological sample initially obtained from a subject prior to administering a gelsolin treatment to the subject is determined to be higher than the amount of MPs comprising the IL-1β, Ly6G, and/or CD66b in a sample obtained subsequent to the gelsolin treatment, it indicates an efficacy of the gelsolin agent to treat and reduce the severity of the signature MP-associated disease or condition in the subject.

Methods of the invention, in some embodiments, include administering a gelsolin agent to a subject who has or is at risk of a signature MP-associated disease or condition in an amount effective to result in a therapeutic effect to reduce the severity of the signature MP-associated disease or condition in the subject. The gelsolin agent can be administered in conjunction with other treatments selected in a therapeutic regimen for a subject identified as having or being at risk of a signature MP-associated disease or condition.

Methods and compositions of the invention may be used to treat a signature MP-associated disease or condition. As used herein, the terms “treat”, “treated”, or “treating” when used in relation to a signature MP-associated disease or condition may refer to a prophylactic treatment that decreases the likelihood or risk of a subject developing the signature MP-associated disease or condition, and may be used to refer to a treatment after a subject has developed a signature MP-associated disease or condition in order to eliminate or ameliorate the signature MP-associated disease or condition, prevent the signature MP-associated disease or condition from becoming more advanced or severe, and/or to slow the progression of the signature MP-associated disease or condition compared to the progression of the signature MP-associated disease or condition in the absence of a therapeutic method of the invention.

### Subjects and Samples

As used herein, a subject may be a vertebrate animal including but not limited to a human, mouse, rat, guinea pig, rabbit, cow, dog, cat, horse, goat, and non-human primate, e.g., monkey. A subject may be a mammal. In some embodiments, a subject is any human or non-human recipient of the inhibitors, compounds or pharmaceutical compositions thereof described herein. In certain aspects of the invention, a subject may be a domesticated animal, a wild animal, or an agricultural animal. Thus, the invention can be used to treat signature MP-associated diseases or conditions in human and non-human subjects. For instance, methods and compositions of the invention can be used in veterinary applications as well as in human treatment regimens. In some embodiments of the invention, a subject is a human. In some embodiments of the invention, a subject has or is at risk of having a signature MP-associated disease or condition and is in need of treatment.

As used herein, a biological sample may be a cell sample, tissue sample, blood sample, bodily fluid sample, saliva sample, sputum sample, nasal secretion sample, amniotic fluid sample, vitreous humor sample, tear sample, urine sample, lymph sample, spinal fluid sample, etc. A biological sample may include cells, tissues, or organelles and may include cell types such as but not limited to: muscle cells, cardiac cells, circulatory cells, neuronal cells, glial cells, fat cells, lung cells, skin cells, hematopoietic cells, epithelial cells, sperm, oocytes, muscle cells, adipocytes, kidney cells, hepatocytes, pancreas cells, etc.

### Assessments and Controls

A signature MP-associated disease or condition in a subject can be detected using a method of the invention. In some embodiments of the invention art-known methods, including but not limited to: assessing one or more characteristics of the signature MP-associated disease or condition such as, but not limited to: presence of the symptoms of the disease or condition may be used in conjunction with methods of detecting a signature MP-associated disease or condition in a subject. Methods of the invention may in some instances include determining a level of severity of a signature MP-associated disease or condition in a subject. Non-limiting examples of ways to determine severity include one or more of: an assay, for example but not limited to a blood gas assay; observing the subject; assessing one or more physiological symptoms exhibited by the subject; assessing the exposure and or activity history of the subject; and assessing a likelihood of survival of the subject. Non-limiting examples of physiological symptoms that may be observed or monitored to assess severity of a reduced oxygenation-associated disease or condition in a subject are: shortness of breath, low blood oxygen saturation, dizziness, muscle pain, organ pain, lung pathology or damage, loss of consciousness, impaired breathing, headache, vascular permeability, and symptoms of poisoning. Non-limiting examples of assessments of exposure and/or activity of a subject include: determining the subject's exposure to significantly high levels of CO₂, determining the subject's exposure to significantly high levels of CO, identifying scuba diving activity of the subject, identifying if the subject was present at high elevation, determining if the subject was exposed to a toxic gas, determining if the subject was exposed to an asphyxiant gas, determining the subject's history of opioid use, and determining if the subject has ingested poison.

Characteristics of a signature MP-associated disease or condition detected in a subject can be compared to control values of the characteristics of the signature MP-associated disease or condition. A control value may be a predetermined value, which can take a variety of forms. It can be a single cut-off value, such as a median or mean. It can be established based upon comparative groups, such as in groups of individuals having the signature MP-associated disease or condition, groups of individuals who have been administered a treatment for the signature MP-associated disease or condition, groups of individuals who have not been administered a treatment for the signature MP-associated disease or condition, etc. Another example of comparative groups may be groups of subjects having one or more symptoms of or a diagnosis of the signature MP-associated disease or condition and groups of subjects without one or more symptoms of or a diagnosis of the signature MP-associated disease or condition. The predetermined value, of course, will depend upon the particular population selected. Accordingly, the predetermined value selected may take into account the category in which an individual falls. Appropriate categories can be selected with no more than routine experimentation by those of ordinary skill in the art.

Controls can be used in methods of the invention to compare characteristics of different control groups, characteristics of a subject with those of a control group, etc. Comparisons between subjects and controls, one control with another control, etc. may be based on relative differences. For example, though not intended to be limiting, a physiological symptom in a subject treated with a gelsolin agent in a therapeutic method of the invention, can be compared to the physiological symptom of a control group that has not been administered the gelsolin agent. The comparison may be expressed in relative terms, for example, if a low blood oxygen level is a characteristic of a signature MP-associated disease or condition, a measurement of blood oxygen level of a subject treated with a therapeutic method of the invention comprising administering a gelsolin agent may be compared to a control level of blood oxygen level. In some embodiments, a suitable control is a subject not treated with a therapeutic method of the invention. A comparison of a treated versus a control may include comparing disease severity differences between the treated subject and the selected control. In some instances, severity of a subject treated with a method of the invention may be determined to be less relative to a selected control, with the comparison indicating up to a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42,%, 43%, 44%, 45%, 46%, 47%, 48%, 49%, 50%, 51%, 52%, 53%, 54%, 55%, 56%, 57%, 58%, 59%, 60%, 61%, 62,%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% reduction in severity of one or more physiological symptoms of the signature MP-associated disease or condition in the subject as compared to the control.

In some embodiments, a level of severity of a treated subject's signature MP-associated disease or condition is less than 100% of a control severity level of the signature MP-associated disease or condition. In certain embodiments of the invention the severity of one or physiological symptoms of the signature MP-associated disease or condition in a subject treated according to a method of the invention is less than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%. 0.9%, 0.8%, 0.7%, 0.6%, 0.5%, 0.4%, 0.3%, 0.2%, or 0.1% of the control level of severity of the one or more physiological symptoms, respectively, of the signature MP-associated disease or condition.

In another non-limiting example, a level of a signature MP-associated disease or condition in a subject and/or increase in a therapeutic effect of administration of a gelsolin agent to the subject using a method of the invention can be determined by comparing a likelihood of survival of the subject treated with a method of the invention with a control likelihood of survival. A non-limiting example of a control likelihood of survival is the likelihood of survival in a subject with the signature MP-associated disease or condition who is not treated with a method of the invention. Non-limiting examples of parameters of likelihood of survival that can be measured include: determination of length of time (hours, days, weeks, etc.) a subject remains alive following a treatment of the invention, and whether a subject dies or survives following a treatment of the invention. It will be understood how these and other parameters relating to likelihood of survival can be compared to controls to assess and determine therapeutic effectiveness of a gelsolin therapeutic method of the invention. A non-limiting example of a control of likelihood of survival is the number of days a subject survives after treatment with a method of the invention compared to the control number of days of survival in the absence of the administration of the effective amount of the gelsolin agent. In some embodiments of the invention a likelihood of survival of a subject treated with a method of the invention is at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 125%, 150%, 175%, 200%, 300%, 400%, 500%, higher than a control likelihood of survival.

It will be understood that controls may be, in addition to predetermined values, samples of materials tested in parallel with the experimental materials. Examples include samples from control populations or control samples generated through manufacture to be tested in parallel with the experimental samples; and also a control may be a sample from a subject prior to, during, or after a treatment with an embodiment of a method or composition of the invention. Thus, one or more characteristics determined for a subject having a signature MP-associated disease or condition may be used as “control” values for those characteristics in that subject at a later time.

### Timing of Administration

Some embodiments of the invention include pretreating a subject who, at the time of treatment, does not have a signature MP-associated disease or condition. In some embodiments, pretreatment of a subject occurs at a time in advance of the subject undertaking an activity or having a potential exposure that puts the subject at risk of developing a signature MP-associated disease or condition. Some embodiments of treatment methods of the invention include administering to a subject having at risk of having a signature MP-associated disease or condition an effective amount of a gelsolin agent, wherein the gelsolin agent is administered from just prior to the activity or potential exposure or up to 1, 2, 3, 4, 5, 6, 12, 18, 24, 48, 72, 96, 120, 144 hour, or more prior to the activity or potential exposure of the subject. In some embodiments the gelsolin agent is administered to the subject at the time of the activity and/or potential exposure of the subject. In some embodiments the gelsolin agent is administered after the activity or potential exposure of the subject. In some embodiments, a subject received a gelsolin agent in a therapeutic method of the invention at two or three of: prior to, during, and after the activity of potential exposure of the subject. It will be understood that a subject identified as having a disease or condition who might at some point develop a signature MP-associated disease or condition may be administered gelsolin as a prophylactic treatment to reduce the likelihood of the onset of the signature MP-associated disease or condition in the subject.

### Preparation and Administration of Pharmacological Agents

Methods and compositions of the invention have important implications for subject treatment and also for the clinical development of new therapies. It is also expected that clinical investigators now will use the present methods for determining entry criteria for human subjects in clinical trials. Health care practitioners select therapeutic regimens for treatment based upon the expected net benefit to the subject. The net benefit is derived from the risk to benefit ratio.

The amount of a treatment may be varied for example by increasing or decreasing the amount of gelsolin agent administered to a subject, by changing the therapeutic composition administered, by changing the route of administration, by changing the dosage timing and so on. The effective amount will vary with the particular signature MP-associated disease or condition being treated, the age and physical condition of the subject being treated, the severity of the signature MP-associated disease or condition, the duration of the treatment, the specific route of administration, and like factors are within the knowledge and expertise of the health practitioner. For example, an effective amount can depend upon the degree to which an individual has been exposed to or affected by exposure to a toxic gas or other element or situation that can cause the signature MP-associated disease or condition.

### Effective Amounts

Methods of the invention comprise administering a gelsolin agent in an amount effective to treat a signature MP-associated disease or condition. An effective amount is a dosage of the gelsolin agent sufficient to provide a medically desirable result. Gelsolin agents are pharmacological agents that may be used in certain embodiments of treatment methods of the invention. It should be understood that pharmacological agents of the invention are used to treat or prevent signature MP-associated diseases or conditions, that is, in some embodiments they may be used to treat an existing signature MP-associated disease or condition in a subject and they may also prophylactically used in subjects at risk of developing a signature MP-associated disease or condition. An effective amount is that amount that can lower a risk of, slow or perhaps prevent altogether the development of a signature MP-associated disease or condition in a subject. It will be recognized when the pharmacologic agent is used in acute circumstances, it is used to prevent one or more medically undesirable results that typically flow from such adverse events.

Factors involved in determining an effective amount are well known to those of ordinary skill in the art and can be addressed with no more than routine experimentation. It is generally preferred that a maximum dose of a pharmacological agent of the invention be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of a pharmacological agent of the invention is that amount effective to treat the condition, such as a signature MP-associated disease or condition. In the case of signature MP-associated diseases or conditions the desired response is inhibiting the progression of the signature MP-associated disease or condition and/or reducing the severity and/or the level of the signature MP-associated disease or condition. This may involve only slowing the progression of the signature MP-associated disease or condition temporarily, although it may include halting the progression of the signature MP-associated disease or condition permanently. This can be monitored by routine diagnostic methods known to those of ordinary skill in the art. The desired response to treatment of the signature MP-associated disease or condition also can be preventing the onset of the signature MP-associated disease or condition.

### Pharmaceutical Agents and Delivery

The pharmacological agents used in the methods of the invention are preferably sterile and contain an effective amount of gelsolin agent for producing the desired response in a unit of weight or volume suitable for administration to a subject. Doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent may be adjusted by the individual health-care provider or veterinarian, particularly in the event of any complication. A therapeutically effective amount typically varies from 0.01 mg/kg to about 1000 mg/kg, from about 0.1 mg/kg to about 200 mg/kg, or from about 0.2 mg/kg to about 20 mg/kg, in one or more dose administrations daily, for one or more days. Gelsolin agents may also be referred to herein as pharmacological agents.

Some embodiments of methods of the invention, comprise a method for treating a method for treating decompression sickness in an individual (referred to interchangeably herein as a subject) in need of such treatment, and the treatment comprises the step of: administering to the individual a therapeutically effective amount of a gelsolin or an analogue thereof. In a non-limiting example, gelsolin (also referred to herein as a gelsolin agent) is administered in a dose from about 3 mg/kg to about 24 mg/kg. In some embodiments, the gelsolin is administered intravenously. Administering gelsolin inhibits a production of microparticles of gas in a blood or a tissue of the individual suffering from decompression sickness. A representative example of form of gelsolin is recombinant gelsolin.

Some embodiments of methods of the invention, comprise a method for a prophylactic treatment of an individual susceptible to an occurrence of decompression sickness, comprising the step of: administering to the individual a therapeutically effective amount of gelsolin or an analogue thereof. In some embodiments, the gelsolin agent is administered in a dose from about 3 mg/kg to about 24 mg/kg.

Various modes of administration are known to those of ordinary skill in the art which effectively deliver the pharmacological agents of the invention to a desired tissue, cell, or bodily fluid. The manner and dosage administered may be adjusted by the individual healthcare practitioner or veterinarian, particularly in the event of any complication. The absolute amount administered will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

Pharmaceutically acceptable carriers include diluents, fillers, salts, buffers, stabilizers, solubilizers and other materials that are well-known in the art. As used herein, the term “pharmaceutically acceptable” refers to molecular entities and compositions that do not. produce an adverse, allergic or other untoward reaction when administered to an animal, such as, for example, a human, as appropriate. The preparation of a pharmaceutical composition that contains an inhibitory compound and/or additional drug will be known to those of skill in the art in light of the present disclosure, as exemplified by Remington's Pharmaceutical Sciences, 18th Ed. Mack Printing Company, 1990, incorporated herein by reference.

Exemplary pharmaceutically acceptable carriers are described in U.S. Pat. No. 5,211,657 and others are known by those skilled in the art. In certain embodiments of the invention, such preparations may contain salt, buffering agents, preservatives, compatible carriers, aqueous solutions, water, etc. When used in medicine, the salts may be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Various modes of administration known to the skilled artisan can be used to effectively deliver pharmaceutical composition of the invention that comprises a gelsolin agent to a subject to produce a therapeutic effect against a signature MP-associated disease or condition in the subject. Methods for administering such a composition or pharmaceutical compound of the invention may be topical, intravenous, oral, intracavity, intrathecal, intrasynovial, buccal, sublingual, intranasal, transdermal, intravitreal, subcutaneous, intramuscular and intradermal administration. In some embodiments of the invention a means for administering a composition of the invention is inhalation.

The invention is not limited by the particular modes of administration disclosed herein. Standard references in the art (e.g., Remington, The Science and Practice of Pharmacy, 2012, Editor: Allen, Loyd V., Jr, 22^{nd} Edition) provide modes of administration and formulations for delivery of various pharmaceutical preparations and formulations in pharmaceutical carriers. Other protocols which are useful for the administration of a therapeutic compound of the invention will be known to a skilled artisan, in which the dose amount, schedule of administration, sites of administration, mode of administration (e.g., intra-organ) and the like vary from those presented herein. Other protocols which are useful for the administration of pharmacological agents of the invention will be known to one of ordinary skill in the art, in which the dose amount, schedule of administration, sites of administration, mode of administration and the like vary from those presented herein.

Administration of pharmacological agents of the invention to mammals other than humans, e.g. for testing purposes or veterinary therapeutic purposes, is carried out under substantially the same conditions as described above. It will be understood by one of ordinary skill in the art that this invention is applicable to both human and animal diseases. Thus, this invention is intended to be used in husbandry and veterinary medicine as well as in human therapeutics. A pharmacological agent may be administered to a subject in a pharmaceutical preparation.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A pharmacological agent or composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, pills, lozenges, each containing a predetermined amount of the active compound (e.g., gelsolin). Other compositions include suspensions in aqueous liquids or non-aqueous liquids such as a syrup, elixir, an emulsion, or a gel.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where the moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane.

For the pharmacological agent the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of gelsolin agent or by release of the biologically active material beyond the stomach environment, such as in the intestine.

Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of gelsolin. Gelsolin is delivered to the lungs of a mammal while inhaling and traverses across the lung epithelial lining to the blood stream.

Nasal (or intranasal) delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

Pharmacological agent(s), including specifically but not limited to a gelsolin agent may be provided in particles. The term “particles” as used herein means nano or microparticles (or in some instances larger particles) that can consist in whole or in part of a gelsolin agent as described herein. The particles may contain the pharmacological agent(s) in a core surrounded by a coating, including, but not limited to, an enteric coating. The pharmacological agent(s) also may be dispersed throughout the particles. The pharmacological agent(s) also may be adsorbed into the particles. The particles may be of any order release kinetics, including zero order release, first order release, second order release, delayed release, sustained release, immediate release, and any combination thereof, etc. The particle may include, in addition to the pharmacological agent(s), any of those materials routinely used in the art of pharmacy and medicine, including, but not limited to, erodible, nonerodible, biodegradable, or nonbiodegradable material or combinations thereof. The particles may be microcapsules which contain the gelsolin in a solution or in a semi-solid state. The particles may be of virtually any shape.

Both non-biodegradable and biodegradable polymeric materials can be used in the manufacture of particles for delivering the pharmacological agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The pharmacological agent(s) may be contained in controlled-release systems. The term “controlled release” is intended to refer to any drug-containing formulation in which the manner and profile of drug release from the formulation are controlled. This refers to immediate as well as non-immediate release formulations, with non-immediate release formulations including but not limited to sustained-release and delayed-release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained-release implant may be particularly suitable for treatment of chronic signature MP-associated diseases or conditions and/or chronic risk of developing a signature MP-associated disease or condition. “Long-term” release, as used herein, means that the implant is constructed and arranged to deliver therapeutic levels of the pharmacological agent(s) for at least 7 days, and preferably 30-60 days. Long-term sustained release implants are well-known to those of ordinary skill in the art and include some of the release systems described above.

The invention also contemplates the use of kits. In some aspects of the invention, the kit can include one or more pharmaceutical preparation vial, a pharmaceutical preparation diluent vial, and a gelsolin agent. A vial containing the diluent for the pharmaceutical preparation is optional. A diluent vial may contain a diluent such as physiological saline for diluting what could be a concentrated solution or lyophilized powder of the gelsolin agent. The instructions can include instructions for mixing a particular amount of the diluent with a particular amount of the concentrated pharmaceutical preparation, whereby a final formulation for injection or infusion is prepared. The instructions may include instructions for treating a subject with effective amounts of the gelsolin agent. It also will be understood that the containers containing the preparations, whether the container is a bottle, a vial with a septum, an ampoule with a septum, an infusion bag, and the like, can contain indicia such as conventional markings that change color when the preparation has been autoclaved or otherwise sterilized.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

### Overview

Experiments were performed to evaluate levels of pGSN in plasma from a previously reported human high-pressure exposure study [see for example Moroianu J et al., PNAS 90: 3815-3819, 1993] and to investigate pGSN in a murine DCS model. Studies undertaken demonstrated that pGSN levels decreased with exposure to high pressure and decompression, and rhu-pGSN abrogated vascular injuries in the murine model.

Experiments were performed to assess decompression sickness and to determine whether pGSN administration would ameliorate injuries in a murine decompression sickness (DCS) model. Research subjects were found to exhibit a modest decrease in pGSN level while at high pressure and a profound decrease after decompression. In some studies, the changes were identified as occurring concurrently with elevations of circulating microparticles (MPs) carrying interleukin (IL)-1β. Mice exhibited a comparable decrease in pGSN after decompression along with elevations of MPs carrying IL-1β. Infusion of recombinant human (rhu)-pGSN into mice before or after pressure exposure abrogated these changes and prevented capillary leak in brain and skeletal muscle.

Human and murine MPs generated under high pressure exhibited surface filamentous (F-) actin to which pGSN binds, leading to particle lysis. Additionally, human neutrophils exposed to high air pressure exhibited an increase in surface F-actin that was diminished by rhu-pGSN resulting in inhibition of MP production. The results indicated benefits of administering rhu-pGSN as prophylaxis or treatment for DCS.

### Materials:

Chemicals were purchased from Sigma-Aldrich (St. Louis, MO) unless otherwise noted. Compressed gases were purchased from Air Products and Chemicals, Inc. (Allentown, PA). BioAegis Therapeutics (North Brunswick, NJ) provided rhu-pGSN. Antibodies and flow cytometry reagents are as follows: Anti-actin (Sigma-Aldrich, St. Louis, Mo, cat #A2066),anti-biotin (Sigma, cat #B3640), anti-Ly6G eFluor450 (eBioscience, San Diego, CA, cat #48-5931-82), anti-mouse CD31 BV510 (Becton Dickinson/Pharmingen, BD, San

Jose, CA cat #563089), annexin V-FITC (BD, cat #556419), anti-CD41 PerCP Cy5.5 (BioLegend, San Diego, CA, cat #133918), anti-CD45 Cy7-A (BioLegend, San Diego, CA, cat #103114), anti-gelsolin PE (Abcam, Cambridge, MA, cat #ab109014), anti-IL-1β (Abcam, Cambridge, MA, cat #ab9722), N-(7-nitrobenz-2-oxa-1,3-thiazol-4-yl) phalloidin (Life technologies, cat #N354). Verification of anti-actin as recognizing β-actin and anti-biotin as recognizing biotinylated actin were shown by Western blot and mass spectroscopy in a prior publication [see for example Thom S R et al., J Biol Chem 289: 18831-18845, 2014]. All antibodies for flow cytometry were documented specifically for this usage by the manufacturers and used at the concentrations recommended. Flow cytometry methods are described below, with positive staining determined following the fluorescence-minus-one control test.

### Animals:

All aspects of this study were reviewed and approved by the Institutional Animal Care and Use Committee. C57BL/6J mice (Mus musculus) were purchased from Jackson Laboratories (Bar Harbor, ME). They were housed in the university animal facility with a 12/12 hour light-dark cycle. Housing and all experiments were conducted at 22-24° C. and 40-70% humidity. They all received water ad libitum and were fed Laboratory Rodent Diet 5001 (PMI Nutritional Inc., Brentwood, MO). Mice were left to breathe room air (control) or subjected to 2 hour exposure to 790 kPa (absolute pressure) air as described in previous publications [see for example Thom S R et al., J Appl Physiol 110: 340-351, 2011; Yang M et al., J Appl Physiol 112: 204-211, 2012]. The air flow rate through the chamber assured no CO₂ build-up. In prior studies the role of elevated nitrogen partial pressure was shown to be the critical stressor causing physiological changes and not the mild elevations of oxygen that occurs with transit or achieving 790 kPa air pressure (Yang et al AJP 119: 219, 2015). Where shown in the text mice were injected with a sterile solution of rhu-pGSN (38.4 mg/ml) at a dose of 27 mg/kg IV, or just the carrier buffer, immediately before or following decompression. At 2 hours after decompression, animals were anesthetized and euthanized for blood and tissue collection as described previously [see for example Thom S R et al., J Appl Physiol 110: 340-351, 2011; Yang M et al., J Appl Physiol 112: 204-211, 2012]. Randomization of mice for experimentation was performed by first collecting all mice to be used in a day into a single plastic cage and then randomly selecting an individual mouse for use as the daily control or intervention group. Studies were done over a span of 4 months with acclimatized mice purchased in groups of 6-12 at bi-weekly intervals and used according to a block design where individual blocks represented mice selected as control or pressure-only, and then with further experimentation including infusion of rhu-pGSN only, rhu-pGSN before or after pressure exposures. Data were scored and analyzed in a blinded manner such that the scorer did not know an animal's group assignment. All mice involved in this project were included in data analysis, none were excluded.

Human subjects:

All procedures were completed in accordance with the Declaration of Helsinki and approved by Ethical Committees of organizations involved with this investigation. Participants provided informed, written consent. Plasma samples analyzed in this project had been frozen and stored as part of a recently published study [see for example Brett K D et al., Sci Rep in press: https://doi.org/10.1038/s41598-41019-49924-41591, 2019]. A sub-group from this study included 6 male research subjects (34±1.2 (SE) years old) who in November 2018 were exposed to air at a pressure of 300 kPa, equivalent to 30 meters of sea water (msw) for 35 minutes and then staged decompression following Canadian Forces Standard Air Decompression Tables (DCIEM). Subjects remained sitting at rest with no exertion during the exposures and did not perform any specific tasks. Pressurization and decompression were conducted with filtered, pure air and no breathing masks were used so as to prevent an elevation of CO₂. Data on MPs and IL-1β from these individuals were included in a previous publication [see for example Brett K D et al., Sci Rep in press: https://doi.org/10.1038/s41598-41019-49924-41591, 2019]. Blood samples were obtained 30 minutes prior to pressurization, after 25 minutes at pressure and 2 hours post-decompression. Blood (˜5mL) was drawn into Cyto-Chex BCT test tubes that contain a proprietary preservative (Streck Inc., Omaha, NE), shipped to the senior author's laboratory, and processed as described previously [see for example Brett K D et al., Sci Rep in press: https://doi.org/10.1038/s41598-41019-49924-41591, 2019]. Plasma stored at −80° ° C. after a 15,000 g centrifugation step preceding MP analysis was used for pGSN assays.

For ex vivo human cell studies, heparin-anticoagulated blood (4 ml) was obtained from healthy human volunteers, centrifuged through a two-layer preparation of Histopaque 1077 and 1119 (Sigma) at 400 g for 30 min to isolate neutrophils. The cells were washed in PBS and a concentration of 9×10⁵ neutrophils/ml of PBS+1 mM CaCl₂, 1.5 mM MgCl₂ and 5.5 mM glucose were exposed at room temperature to either air at atmospheric pressure (˜100 kPa) or air at a partial pressure of 790 kPa following published procedures [see for example Thom S R et al., J Biol Chem 289: 18831-18845, 2014].

### Standard Procedures for MP Isolation:

All reagents and solutions used for MP isolation and analysis were filtered with 0.1 μm filter (EMD Millipore, Billerica, MA). MPs were isolated and prepared for analysis by flow cytometry as previously described [see for example Thom S R et al., J Appl Physiol 110: 340-351, 2011; Yang M et al., J Appl Physiol 112: 204-211, 2012]. Briefly, blood was centrifuged for 5 min at 1,500 g. EDTA was added to the supernatant to achieve 12.5 mM to prevent MPs aggregation, and centrifuged at 15,000 g for 30 min. The supernatant was used for MP count and subtypes analysis by flow cytometry as described [see for example Thom S R et al., J Appl Physiol 110: 340-351, 2011; Yang M et al., J Appl Physiol 112: 204-211, 2012], and samples were frozen at −80° C. for later assays of IL-1β and pGSN.

### MP Analysis:

MPs were analyzed as described previously [see for example Thom S R et al., J Appl Physiol 110: 340-351, 2011; Yang M et al., J Appl Physiol 112: 204-211, 2012]. In brief, flow cytometry was performed with an 8-color, triple laser MACSQuant® Analyzer (Miltenyi Biotec Corp., Auburn, CA) using MACSQuantify™ software version 2.5 to analyze data. MACSQuant was calibrated every other day with calibration beads (Miltenyi Biotec Corp., Auburn, CA). Forward and side scatter were set at logarithmic gain. Photomultiplier tube voltage and triggers were optimized to detect sub-micron particles. Micro-beads of 3 different diameters 0.3 μm (Sigma, Inc., St. Louis, MO), 1.0 μm and 3.0 μm (Spherotech, Inc., Lake Forest, IL) were used for initial settings and before each experiment as an internal control. Samples were suspended in Annexin binding buffer solution (1:10 v/v in distilled water, (BD Pharmingen, San Jose, CA), and antibodies as listed. Phalloidin binding was assessed to probe for the presence of F-actin. Examples of blood-borne particles analysis have been published previously [see for example Bhullar J et al., Fr Radic Biol Med 101: 154-162, 2016]. All reagents and solutions used for MP analysis were sterile and filtered (0.1 μm filter). MPs were defined as annexin V-positive particles with diameters of 0.3 to 1 μm diameter. The concentration of MPs in sample tubes was determined by MACSQuant® Analyzer according to exact volume of solution from which MPs were analyzed.

Surface proteins on MPs from control and decompressed mice were biotinylated using sulfosuccinimidyl 2-(biotinamido)ethyl-1,3-dithiopropionate (NHS-SS-biotin) following methods similar to those described by others [see for example Moroianu J et al., PNAS 90: 3815-3819, 1993]. The 15,000 g plasma supernatant described above was centrifugation at 100,000 g for 1 hour and MPs resuspended in PBS without or with 100 mg/ml rhu-pGSN. After 30-minute incubation at room temperature, ice-cold NHS-SS-biotin (0.9 mg/ml) was added and samples incubated on ice for 15 minutes. Biotinylation was quenched by addition of 100 mM glycine and MPs sedimented by centrifugation at 100,000 g for 1 hour. The MP pellets were subjected to Western blotting or the biotinylated proteins separated from MP lysates for analysis.

For Western blots, MPs were resuspended in 100 mM phosphate buffer with 2% sodium dodecyl sulfate (SDS), 10% glycerol, 5% β mercaptoethanol, and 0.00125% bromophenol followed by electrophoresis using a 4-15% gradient polyacrylamide gel (SDS-PAGE), transfer to nitrocellulose paper and proteins probed for biotin, actin and IL-1β. Alternatively, following ultracentrifugation the MP pellets were resuspended in 100 μl lysis buffer (20 mM Tris, 150 mM NaCl, 1% Nonidet P-40, 0.5% sodium deoxycholate, 1 mM EDTA and 0.1% SDS (pH 7.5) with protease inhibitors cocktail (Sigma)), and incubated for 30 minutes on ice. MagVigen®-streptavadin magnetic nanoparticles (Nvigen, Inc., Sunnyvale, CA) were then added and incubated for 12 hours before the biotinylated proteins were separated for Western blotting using a magnet followed by washing and magnetic bead separation steps according to the manufacturer's recommended procedure.

### Vascular Permeability Assay:

Mice were injected with lysine-fixable tetramethylrhodamine-conjugated dextran (2×10⁶ Da, Invitrogen, Carlsbad, CA) and endothelium-enriched tissue homogenates prepared using colloidal silica following published methods [see for example Thom S R et al.,J Appl Physiol 110: 340-351, 2011; Yang M et al., J Appl Physiol 112: 204-211, 2012]. Vascular permeability, quantified as perivascular dextran uptake in the experimental group, was normalized to a value obtained with a control mouse included in each experiment.

### IL-1β Measurements:

Human or mouse-specific ELISA Kits (eBioscience, San Diego, CA) that detect pro-and mature forms of IL-1β were used following the manufacturer's instructions. Measurements were made using plasma supernatant after blood was centrifuged at 15,000 g as described for flow cytometry studies, and also in MPs separated from plasma by centrifugation at 100,000 g for 60 min. The MPs in pellets were placed in 0.3 ml lysis buffer, protein content of the sample was measured, diluted to 5 mg/ml, and 20 μg protein used for analysis.

### Gelsolin Assay:

Human and mouse-specific commercial ELISA kits (LSBio, Inc. Seattle, WA) were used for measuring pGSN following the manufacturer's instructions. Serial dilutions in PBS were prepared using the supernatant after 15,000 g centrifugation of plasma as described above and analyzed concurrent with a range of known pGSN standards.

### Statistical Analysis:

Results are expressed as the mean±SE for three or more independent experiments. Data were compared by t-test or analysis of variance (ANOVA) and Newman-Keuls post-hoc test using SigmaStat (Jandel Scientific, San Jose, CA). Data from human subjects were compare by repeated measures analysis of variance (RM ANOVA) on ranks. For all studies, the level of statistical significance was defined as p<0.05.

### Results

Human Studies and Murine Model—MPs, pGSN and IL-1β:

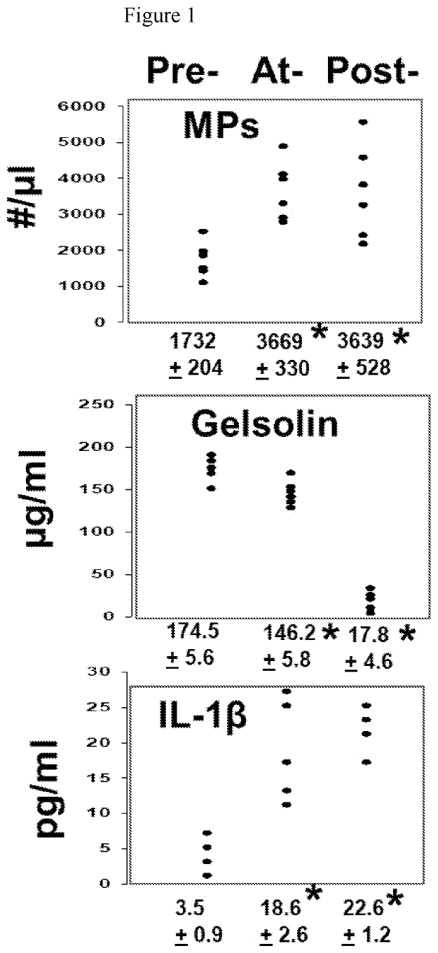

Blood samples from six research subjects were obtained before, during and 2 hours after exposure to 300 kPa air pressure in a hyperbaric chamber. FIG. 1 demonstrates the relationships among MPs, pGSN and plasma IL-1β. Exposure to pressure resulted in statistically significant elevations of MPs and IL-1β, and a decrease in pGSN while at pressure with a further decrease of pGSN levels after decompression.

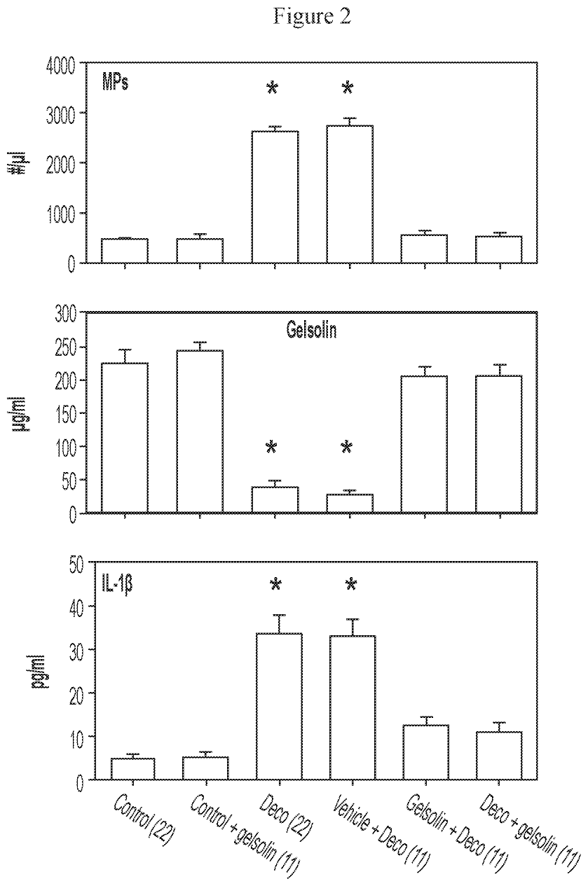

The impact in mice of exposure to 790 kPa air pressure for 2 hours on the number of circulating MPs, pGSN and IL-1β is shown in FIG. 2. Statistically significant changes were found with elevations of MPs and plasma IL-1β concurrent with a decrease in pGSN. These changes were abrogated when mice were injected intravenously with rhu-pGSN immediately before pressurization or after decompression. Infusion of the carrier buffer used for rhu-pGSN injections had no significant effect on pressure responses, and infusions of rhu-pGSN into air-exposed control mice caused no statistically significant changes.

IL-1β secretion requires unconventional pathways, and a major route involves packaging into a vesicle to be liberated to the extracellular milieu [see for example Cypryk W et al., Front Immunol 9: 2188, 2018]. The intra-MP IL-1β concentrations expressed as pg/million MPs among the six human subjects were 24.5±5.4 (SE) pre-pressure, 98.2±17.5 at pressure, and 126.9±20.8 post-decompression (p<0.05 among all three by RM ANOVA). A similar relationship in mice pre- versus post-decompression was observed (Table 1). Prophylactic rhu-pGSN administration did not prevent the increase in intra-particle IL-1β concentration whereas treatment post-decompression did abrogate the elevation (see 5^{th} and last lines in Table 1).

TABLE 1
Murine IL-1β/million MPs. Data shown the concentration
of intra-MPs IL-1β (pg/million MPs) as mean +
SE obtained from male mice manipulated as described in
the caption for FIG. 2. The (n) for each group is shown,
* indicates significantly different from control, p < 0.05, ANOVA.

| Group | pg/million MPs |
|---|---|
| Control (22) | 10.2 ± 1.2 |
| Control + pGSN (4) | 9.6 ± 1.3 |
| Deco (11) | 35.2 ± 6.1* |
| Vehicle + Deco (4) | 33.8 ± 5.2* |
| pGSN + Deco (4) | 47.9 ± 6.8* |
| Deco + pGSN (4) | 13.9 ± 4.0 |

### Murine Model—Vascular Permeability:

Studies were performed to evaluate whether rhu-pGSN had an effect on tissue injury in the decompression model. Vascular permeability to rhodamine-labeled dextran was significantly elevated in skeletal muscle and brain at 2 hours after decompression (Table 2). Vascular leakage was abrogated in mice that received rhu-pGSN prior to pressurization or immediately after decompression. Permeability was not significantly different from control when normal air-exposed mice were injected with rhu-pGSN.

TABLE 2
Murine vascular leakage of 2 × 10⁶ Da rhodamine-labeled
dextran. Extravasation of dextran in brain and leg skeletal
muscle was evaluated as described in Methods in mice manipulated
as described in the caption for FIG. 2. Data are fold-difference
in rhodamine-dextran/mg tissue protein (mean + SE) versus
the values in control mice processed concurrently with each
experimental group. Sample number is indicated as (n), * indicates
significantly different from control, p < 0.05, ANOVA.

| | Brain | Muscle |
|---|---|---|
| Control + pGSN (4) | 1.1 ± 0.1 | 1.1 ± 0.1 |
| Deco (6) | 4.8 ± 1.4* | 2.9 ± 1.5* |
| Vehicle + Deco (6) | 4.6 ± 0.9* | 2.3 ± 0.7* |
| pGSN + Deco (4) | 1.3 ± 0.2 | 1.1 ± 0.2 |
| Deco + pGSN (4) | 1.4 ± 0.2 | 1.0 ± 0.2 |

### MP Surface Protein Expression Patterns:

MP sub-types were characterized based on expression of surface proteins. As in past studies, higher numbers of each sub-type were found in decompressed mice [see for example Thom S R et al., J Appl Physiol (1985) 125: 1339-1348, 2018; Thom S R et al., J Appl Physiol 112: 1268-1278, 2012; Thom S R et al., J Appl Physiol 114: 1396-1405, 2013; Thom S R et al., J Appl Physiol 110: 340-351, 2011]. Values can be derived by multiplying total MP numbers by the % of each subtype shown in Table 3. However, it was noted that strictly looking at % of each type offered insight into differences in possible cell sources for MPs. Table 3 demonstrates statistically significant differences from control in fractions of MPs expressing Ly6G (a neutrophil membrane protein) and those with a pattern consistent with endothelial cells (based on expression of CD31 [platelet-endothelial cell adhesion protein], but null for CD41 [a component of platelet-specific β₃ adhesion molecule]) from decompressed mice and decompressed mice injected with the carrier buffer. Among mice administered rhu-pGSN before or after decompression, the fraction expressing Ly6G was again significantly different from control, in contradistinction to the sub-type expressing endothelial cell markers which was nearly at the control level. Hence, administration of rhu-pGSN prevented generation of endothelial-derived MPs in response to decompression. As expected based on prior reports, when one adds up all the sub-types the total exceeds 100%, likely indicating that surface proteins are shared among MPs due to collisions in the blood stream [see for example Thom S R et al., J Appl Physiol 110: 340-351, 2011].

Microparticles binding of phalloidin as an index of F-actin was also examined, given that one biochemical action of pGLN is to cleave F-actin [see for example Fu L et al., Front Immunol 8: 917, 2017; Ljubkovic M et al., J Appl Physiol 109: 1670-1674, 2010]. As shown, the fraction of microparticles that bound phalloidin increased 8-fold in decompressed mice. Notably, phalloidin binding by microparticles was not significantly different from control among mice injected with pGSN.

TABLE 3
MPs sub-types in mice. Blood-borne MPs were quantified in mice manipulated as
described in the caption for FIG. 2. Flow cytometric measurements were made to quantify the
number of all 0.3 to 1 μm diameter Annexin V-positive particles (data in FIG. 2) as well as
the fraction of those expressing proteins specific to certain cells [Ly6G (mature neutrophils),
CD14 (all leukocytes), CD31 (platelets and endothelium), CD41 (platelets), CD31+/CD41−
dim (endothelium, labeled ECs)] and also those that bound phalloidin (Phall). Data are
mean + SE, n is shown for each sample, * indicates significantly different from control,
p < 0.05, ANOVA.

| | % | % | % | % | % | % |
|---|---|---|---|---|---|---|
| | Ly6G | CD14 | CD31 | CD41 | ECs | Phall. |
| Control (22) | 0.1 ± 0.1 | 44.3 ± 4.1 | 47.8 ± 5.7 | 29.1 ± 3.7 | 0.6 ± 0.1 | 2.3 ± 0.6 |
| Control + pGSN | 1.0 ± 0.5 | 45.9 ± 5.1 | 36.1 ± 4.9 | 23.6 ± 4.4 | 0.8 ± 0.2 | 3.2 ± 0.7 |
| (11) | | | | | | |
| Deco (11) | 11.1 ± 1.9* | 51.2 ± 3.5 | 36.8 ± 3.4 | 18.0 ± 3.3 | 2.3 ± 0.3* | 29.4 ± 3.3* |
| Vehicle + Deco | 14.3 ± 2.5* | 47.1 ± 3.3 | 49.4 ± 4.5 | 19.7 ± 3.4 | 1.7 ± 0.2* | 24.5 ± 2.5* |
| (11) | | | | | | |
| pGSN + Deco | 3.9 ± 0.9* | 50.6 ± 3.2 | 38.9 ± 3.6 | 21.1 ± 3.5 | 0.4 ± 0.1 | 4.5 ± 1.0 |
| (11) | | | | | | |
| Deco + pGNS | 5.3 ± 0.8* | 54.9 ± 6.8 | 40.2 ± 8.1 | 30.0 ± 5.7 | 0.5 ± 0.1 | 6.2 ± 2.0 |
| (11) | | | | | | |

### Actin Presence on the MP Membrane:

The loss of MPs in decompressed mice injected with rhu-pGSN could indicate that F-actin may be its target on the particle surface, given that one biochemical action of pGSN is to bind and then cleave F-actin [see for example Lee P S et al., Am Soc Nephrol 20: 1140-1148, 2009; Ordija C M et al., Am J Physiol Lung Cell Mol Physiol 312: L1018-L1028, 2017]. To investigate this possibility, flow cytometry was used to evaluate whether fluorescently labeled phalloidin would bind to MPs. As shown in Table 3, the fraction of MPs that bound phalloidin increased 8-fold in decompressed mice. Phalloidin binding by MPs was not significantly different from control among decompressed mice injected with rhu-pGSN.

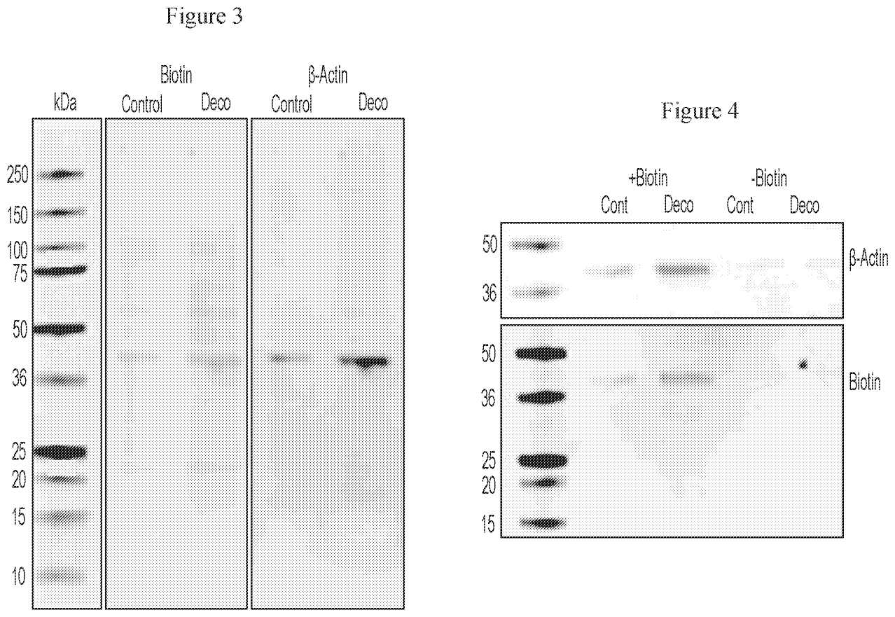

Supporting evidence to assess whether actin was present on the MP surface was sought by selective surface protein biotinylation using NHS-SS-biotin (see Methods herein). FIG. 3 is a representative Western blot of four showing that the prominent 43 kDa biotinylated protein band is also recognized by anti-β-actin. In replicate experiments the 43 kDa protein band of MPs from decompressed mice was 2.9±0.3 -fold denser than the band with control MPs (n=4, p<0.05). When incubated with 200 μg/ml rhu-pGSN (comparable to that of normal plasma-see FIG. 1) the band density of control MPs was reduced by 26.3+4.3% whereas with MPs from decompressed mice the band density was decreased by 61.1+3.2% (p<0.05). No biotinylated protein bands were seen at 17 or 31 kDa where mature and pro-IL-1β respectively are located, nor were bands detected when Western blots were probed for IL-1β. IL-1β has been reported to be present inside but not adsorbed to the surface of MPs from decompressed mice [see for example Thom S R et al., J Appl Physiol (1985) 125: 1339-1348, 2018]. Therefore, NHS-SS-biotin labeled membrane surface proteins and did not gain access to internal MP proteins.

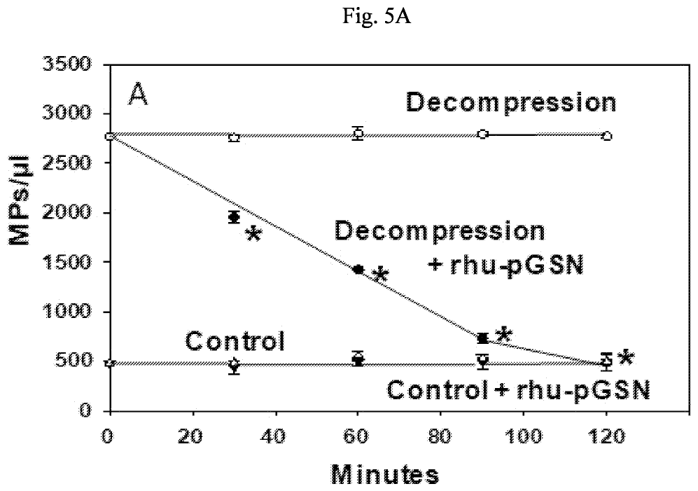

Biotinylated proteins were also separated from non-biotinylated proteins for analysis. FIG. 4 shows a representative Western blot using lysates from biotinylated MPs isolated from control and decompressed mice probed for biotin and β-actin. In four replicates, no IL-1β was detected. Further, the results demonstrate that the majority of MP β-actin is present on the membrane surface and only scant amounts were detected in the biotin-negative MPs.

Ex Vivo Studies of rhu-pGSN Incubations With Murine MPs:

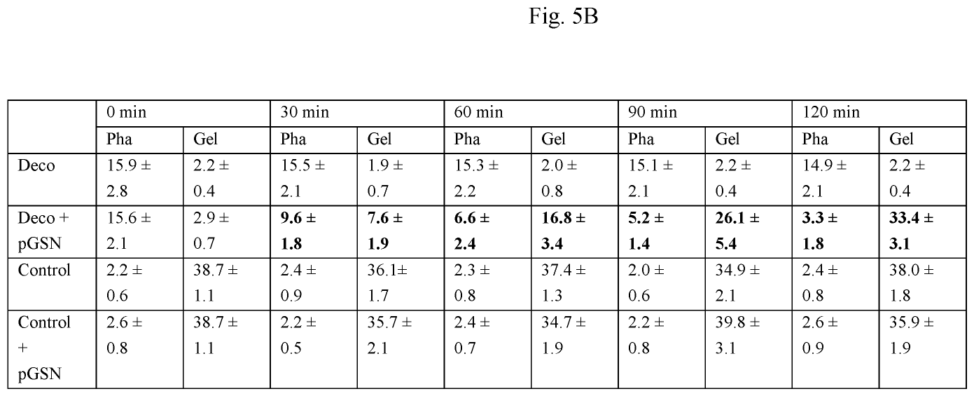

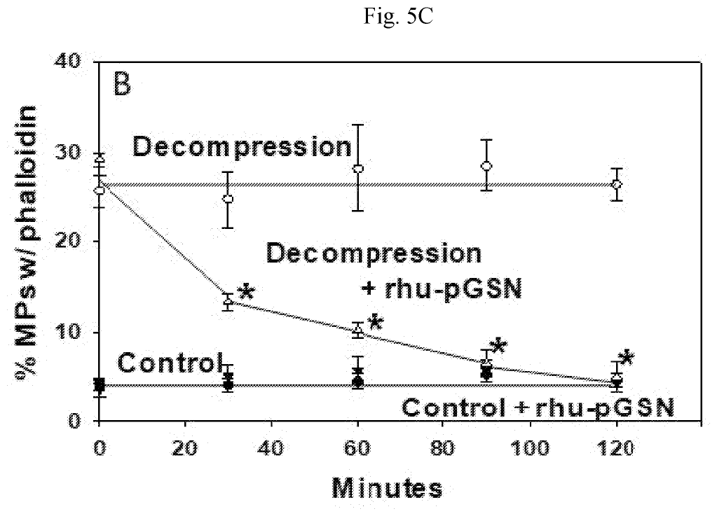

When MPs isolated from control and decompressed mice were suspended in buffer, particle numbers were stable over a 2-hour ex vivo incubation (FIG. 5A). Blood was obtained from control or decompressed male mice and centrifuged as described in Methods. MPs suspensions were divided and where shown at time 0, 200 μg/ml rhu-pGSN was added. At 30 minute intervals samples were fixed. The number of remaining MPs are shown in FIG. 5A. FIG. 5B shows the % of MPs that bind anti-gelsolin antibody and phalloidin. Only values in dark shaded boxes are statistically significantly different from the values as time 0 (p<0.05, ANOVA). However, if rhu-pGSN was added to suspensions, the MPs from decompressed but not control mice were lysed. After each sample was fixed, fluorescent phalloidin and a fluorophore-labeled antibody that recognizes mouse and human pGSN were added to evaluate particle surface F-actin and pGSN binding. The FIG. 5C plot shows that the fraction binding phalloidin decreased for only the MPs from decompressed mice incubated with rhu-pGSN. Surface-bound pGSN values for the four groups were: 11.5±1.8% for control MPs, 12.1±3.3% (NS) for control MPs where rhu-pGSN was added, 15.2±3.6 (NS) for MPs from decompressed mice, and 26.6±4.4 (p<0.05, ANOVA) for MPs from decompressed mice where rhu-pGSN was added. These values did not change significantly over the course of the 2-hour study.

Gelsolin can diminish phalloidin binding to F-actin due to F-actin cleavage and also because of displacement events [see for example Allen PG et al., J Biol Chem 269: 32916-32923, 1994; Kinosian H J et al., Biochemistry 35: 16550-16556, 1996]. Results of experiments described herein demonstrated that the kinetics of MP lysis and pGSN binding were not changed when experiments were done in the presence of equal concentrations of non-fluorescent and fluorescent phalloidin (data not shown), suggesting that the reduction in fluorescent phalloidin bound to decompressed MPs was due to F-actin cleavage.

Ex Vivo Studies of rhu-pGSN Incubations With Human Neutrophils:

In certain studies, microparticles from control and post-decompression mice were isolated and suspended in buffer, resulting in stable particle numbers over a 2-hour ex vivo incubation (FIG. 5). However, if pGLN was added to suspensions at a concentration of 200 μg/ml (comparable to that of plasma-see FIG. 1) those from decompressed mice were lysed. After each sample was fixed, fluorescent phalloidin and a fluorophore-labeled antibody to gelsolin were added to evaluate particle surface F-actin and pGLN binding. Changes were nominal in both control and post-decompression samples without added pGLN, but the presence of phalloidin and gelsolin changed in opposite directions with the decompressed microparticles incubated in the presence of pGLN.

Effects of rhu-pGSN on human neutrophils were examined because prior studies have shown that neutrophils play a major role in MP generation and vascular damage in the DCS model [see for example Thom S R et al., J Appl Physiol 119: 427-434, 2015; Thom S R et al., J Appl Physiol (1985) 126: 1006-1014, 2019; Thom S R et al., J Appl Physiol (1985) 125: 1339-1348, 2018; Thom S R et al., J Appl Physiol 110: 340-351, 2011]. It was determined that that when human cells are incubated at high gas pressure MP production is maximal in 30 minutes with no further production whether cells remain at pressure or they are decompressed [see for example Thom S R et al., J Biol Chem 289: 18831-18845, 2014]. Human neutrophils (1.5×10⁵ in 200 μl buffer) generated 1885±139 (SE, n=10) MPs/μl over 30 minutes when exposed to 790 kPa air pressure. If cells were incubated at 790 kPa in the presence of 200 μg/ml rhu-pGSN significantly fewer MPs, 657±93/μl (n=10, p<0.05) were produced. Cell suspensions incubated in air at ambient pressure had 493±71 MPs/μl at the start of incubations and 538±52 MPs/μl at the end (not significantly different) and numbers were unchanged in the presence of rhu-pGSN.

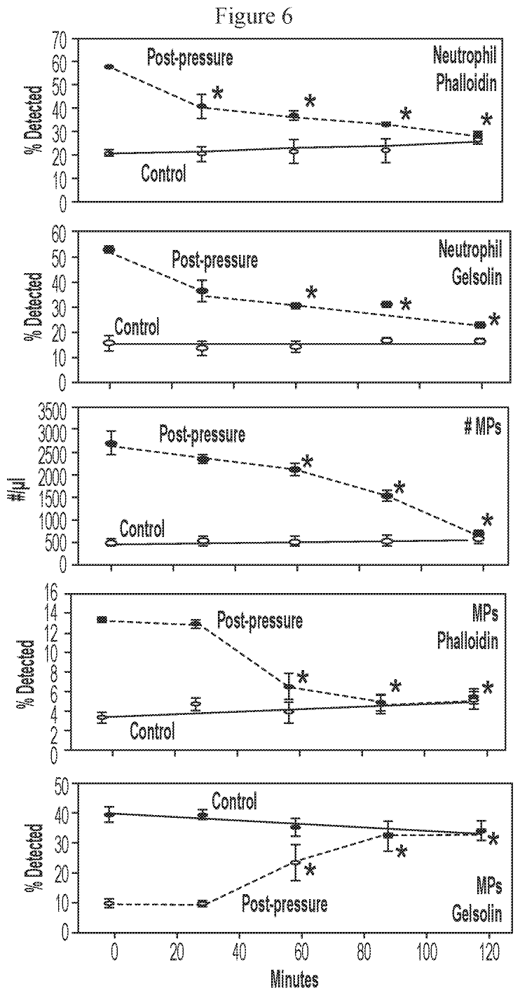

Neutrophil suspensions were investigated in studies in which the suspensions were first incubated in air at ambient pressure or at 790 kPa for 30 minutes and rhu-pGSN added to each post-pressure. Time 0 in FIG. 6 indicates addition of 200 μg/ml rhu-pGSN. At 30-minute intervals the cells and MPs in samples were fixed, separated by centrifugation and analyzed by flow cytometry. While rhu-pGSN had no effect on neutrophil number or viability (data not shown), it did impact surface staining pattern of decompressed cells. The first plot in FIG. 6 demonstrates the fraction of neutrophils that stained with fluorescent phalloidin. Control cells exhibited relatively low phalloidin binding and no significant change with time. Phalloidin binding on cells first subjected to pressure was significantly different from control but decreased with time in the presence of rhu-pGSN. The second plot in FIG. 6 shows neutrophil staining with gelsolin antibody. Again, control cells exhibited relatively low staining and no change with time. However, cells that had been exposed to high pressure had significantly more gelsolin antibody staining and values decreased over 2-hours in parallel with the drop in phalloidin binding.

The next three rows in FIG. 6 show data pertaining to the MPs present in the suspensions. Addition of rhu-pGSN to control preparations did not alter the number of MPs, phalloidin binding, or gelsolin antibody binding. In pressure-exposed suspensions where rhu-pGSN was added, the number of MPs and fraction with high phalloidin binding decreased significantly with time, while the fraction staining with gelsolin antibody increased. Note that gelsolin antibody binding started out rather high in control samples. These were microparticles present in plasma when neutrophils were first removed from blood, because microparticles are not generated by cells when exposed to air at ambient pressure. Contrary to this, microparticles generated by pressure-exposed neutrophils that had been suspended in buffer exhibited increased gelsolin antibody binding over the 2-hour incubation time.

### Discussion

Exposure to high pressure decreases pGSN in blood of humans and mice concurrently with elevations of MPs and IL-1β (FIGS. 1 and 2). In mice, MPs containing high concentrations of IL-1β are responsible for causing diffuse capillary leak [see for example Thom S R et al., J Appl Physiol (1985) 126: 1006-1014, 2019; Thom S R et al., J Appl Physiol (1985) 125: 1339-1348, 2018]. Surface proteins expressed on MPs in decompressed mice exhibited significantly more CD31+/CD41-dim, consistent with endothelial activation/damage, as well as Ly6G, indicative of a neutrophil origin (Table 3). Administration of rhu-pGSN to mice before or after pressure/decompression prevented elevations in the total number of MPs, the IL-1β concentration in plasma, the MPs sub-set from endothelium, and capillary leakage (FIG. 2, Table 2). When rhu-pGSN was administered prophylactically intra-MP IL-1β concentration was elevated after decompression, whereas rhu-pGSN treatment post-decompression resulted in an intra-MP IL-1β concentration that was not significantly different from control (Table 1). The results indicated these differences could be explained by the data evaluating the impact of rhu-pGSN on MPs and neutrophils ex vivo.

One biochemical action of pGSN is to bind and then cleave F-actin, a process which is thought to abrogate intravascular injuries and organ damage [see for example Lee P S et al., Am Soc Nephrol 20: 1140-1148, 2009; Ordija C M et al., Am J Physiol Lung Cell Mol Physiol 312: L1018-L1028, 2017]. Others have shown a complementary relationship between circulating F-actin and pGSN levels, the presence of pGSN-actin complexes in plasma, and depletion of circulating pGSN with local sequestration at injured sites [see for example Khatri N et al., J Diab Res 2014: 152075: 2014; Lee P S et al., Am Soc Nephrol 20: 1140-1148, 2009; Lind S E et al., Am Rev Respir Dis 138: 429-434, 1988; Lu C-H et al., Arch Biochem Biophys 529: 146-156, 2013]. FIGS. 3 and 4 show that actin was present on the MP membrane surface, especially those from decompressed mice, and phalloidin binding (Table 3, FIG. 5) indicated the presence of F-actin. Similarly, MPs produced by high gas pressure stimulated human neutrophils ex vivo also exhibited high phalloidin binding (FIG. 6). When rhu-pGSN was added to murine or human MP suspensions it bound preferentially to pressure-generated MPs, and MPs lysed as the fraction binding phalloidin dropped. Therefore, the results of experiments described herein suggested pGSN was binding to F-actin and cleavage rendered the MPs sensitive to osmotic lysis.

The inverse relationship between circulating pGSN and MPs in humans and mice with pressure exposure (FIGS. 1 and 2) were interpreted as arising because pGSN binds to the increasing number of MPs. Moreover, Table 3 demonstrates that the fraction of MPs binding phalloidin in decompressed mice injected with rhu-pGSN was not significantly different from control. This observation suggested that MPs lysis was selective and rhu-pGSN did not destroy MPs exhibiting low phalloidin binding. The same relationship was seen with ex vivo murine MPs in FIG. 5 and human MPs in FIG. 6. Rhu-pGSN lysed the phalloidin-positive MPs, leaving the same number of MPs in the preparations after the 2-hour incubations as were present in the control samples. However, phalloidin binding was not a quantitative index of susceptibility for lysis by rhu-pGSN. Approximately 20% of post-pressure murine MPs in FIGS. 5, and 14% in FIG. 6 exhibited phalloidin binding at time 0, and the fraction dropped to about 4% over the 2-hour studies. In this same time period, the total number of MPs dropped by ˜80% (from 2600-2800/μl to about 500-520/μl). This difference may occur because F-actin binding on some MPs is below the threshold of detection by flow cytometry or because of additional pGSN ligands such as anionic phospholipids on MPs.

Actin has been detected on the membrane surface of platelets, neutrophils, monocytes, lymphocytes, endothelial cells and sympathoadrenal/catecholaminergic cells [see for example Dudani A K et al., Br J Haematol 95: 168-178, 1996; Fu L et al., Front Immunol 8: 917, 2017; Miles L A et al., J Neurosci 26: 13017-13024, 2006; Pardridge W M et al., J Cereb Blood Flow Metab 9: 675-680, 1989; Por S B et al., J Histochem Cytochem 39: 981-985, 1991; Smalheiser N R, Proteins in unexpected locations. Mol Biol Cell 7: 1003-1014, 1996]. A recent study found that macrophage MPs generation requires extracellular F-actin, which appears to influence caspase-1 activation at filopodia [see for example Rothmeier A S et al., J Clin Invest 125: 1471-1484, 2015]. Results of experiments described herein indicated that approximately 80% of human neutrophils exposed to high gas pressure ex vivo exhibited phalloidin binding versus just 20% of control cells (FIG. 6). High pressure inert gases stimulate neutrophils by triggering oxidative stress [see for example Thom S R et al., J Biol Chem 289: 18831-18845, 2014], and it now appears that F-actin expression on the cell surface is associated with this process. It seems reasonable that extracellular F-actin is transferred to the newly generated MPs budding from the cell surface in response to pressure, explaining why pressure-generated MPs exhibit higher phalloidin binding and why rhu-pGSN selectively impacts decompressed mouse MPs versus the MPs of control mice (FIGS. 2 and 5) and pressure-generated human MPs (FIG. 6).

Results of studies described herein demonstrated that gelsolin antibody binding controlled mouse and human MPs. With regard to the human neutrophil studies (FIG. 6), MPs were not generated during incubations at ambient pressure, so the MPs present in control samples were carried through from plasma. The control MPs appear to have only scant F-actin as they exhibited relatively low phalloidin binding (˜3.5%) but pGSN appears to be present on ˜40% of the MPs. There could be an alternative mechanism for pGSN binding other than F-actin. pGSN has a high affinity for binding to fibronectin. Others have shown that pGSN cell attachment can be mediated via soluble fibronectin which will attach to cell membranes via integrins and glycoproteins [see for example Bohgaki M et al., J Cell Mol Med 15: 141-151, 2011; Giancotti F G et al., J Cell Biol 103: 429-437, 1986].

FIG. 6 also shows that rhu-pGSN cleaves F-actin on the post-decompression neutrophil surface, as demonstrated by the drop in phalloidin binding. Binding by the pGSN antibody decreased in parallel, suggesting that as F-actin is cleaved, pGSN could no longer bind to the neutrophil membrane. Additionally, it was found that inclusion of rhu-pGSN with human neutrophils while exposed to high pressure inhibited MPs production by ˜65% (1885±139 MPs/μl versus 657±93/μl). Thus, surface F-actin may be needed for MPs generation in response to gas pressure. This reflected a separate action in addition to direct MPs lysis, and the effect could be the basis for differences noted in intra-MPs IL-1β concentration between mice infused with rhu-pGSN prophylactically versus injection after decompression (see Table 1). Administration post-decompression destroyed virtually all pressure-induced MPs, including the ones carrying high IL-1β, whereas prophylactic rhu-pGSN administration impeded but did not entirely prevent MPs generation.

Results from this study highlighted the role of MPs as a cytokine carrier. IL-1β was cleared from the plasma within 2 hours after injection of rhu-pGSN in decompressed mice (FIG. 2). Simply lysing MPs would not immediately diminish the plasma concentration of IL-1β but lysis abrogated capillary leak mediated by IL-1β [see for example Thom S R et al., J Appl Physiol (1985) 126: 1006-1014, 2019; Thom S R et al., J Appl Physiol (1985) 125: 1339-1348, 2018]. Hence, MPs appear to have an important role targeting IL-1β to endothelium. This is an area that remains poorly understood and worthy of future research. Results of studies described herein suggest that supplementation with rhu-pGSN can prevent or reverse DCS by reducing inflammatory MPs. This represents a new action for rhu-GSN that may have relevance to a broad number of inflammatory injuries.

### Example 2

A biological sample comprising blood is obtained from a subject and microparticles are detected in the sample. The detected microparticles are examined to determine the presence or absence of microparticles comprising an IL-1β signature, a lymphocyte antigen 6 complex locus G6D (Ly6G) signature, or a CD66b signature.

The presence of an IL-1β signature is detected in the sample, confirming the presence of a signature MP-associated disease or condition in the subject from whom the biological sample is obtained. Based at least in part of the finding of the IL-1β signature, a therapeutic regimen is selected for the subject to treat the signature MP-associated disease or condition. The therapeutic regimen is administered to the subject.

### Example 3

A subject is identified as having a signature MP-associated disease or condition and the subject is administered an effective amount of a gelsolin agent as a treatment for the signature MP-associated disease or condition. The gelsolin agent is effective in treating the signature MP-associated disease or condition in the subject.

### Example 4

A signature MP-associated disease or condition is prevented in a subject. A subject at risk of exposure to an event or environmental condition that puts the subject at increased risk of a signature MP-associated disease or condition is administered an effective amount of a gelsolin agent to reduce the risk and/or severity of the signature MP-associated disease or condition in the subject compared to a control risk, such as, but not limited to the subject's risk in the absence of the administered gelsolin agent. The gelsolin agent is administered to the subject one or more of: prior to, during, and after the subject's exposure to the event or environmental condition. In some studies, the event comprises scuba diving. In some studies the environmental condition comprises exposure to carbon monoxide or other gas that puts the subject at risk of a signature MP-associated disease or condition.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1. A method of determining the presence of a signature MP-associated disease or condition in a subject, comprising:
(a) detecting in a biological sample obtained from a subject suspected of having a signature MP-associated disease or condition, the presence of microparticles;
(b) identifying the detected microparticles as comprising an IL-1β signature, a lymphocyte antigen 6 complex locus G6D (Ly6G) signature, or a CD66b signature; wherein the identification of the IL-1β, Ly6G, or CD66b signature confirms the presence of the signature MP-associated disease or condition in the subject;
(c) selecting a therapeutic regimen for the subject based at least in part on the confirmation of the presence of the signature MP-associated disease or condition in the subject; and
(d) administering the selected therapeutic regimen to the subject to treat the signature MP-associated disease or condition.

2. The method of claim 1, wherein the IL-1β signature, the Ly6G signature, and the CD66b signature are based on: (1) the presence in the biological sample of the MPs comprising one or more of IL-1β, Ly6G, and CD66b, respectively; and (2) the number of MPs comprising one or more of IL-1β, Ly6G, and CD66b, respectively, relative to the total number of MPs in the biological sample.

3. The method of claim 1, further comprising determining in the biological sample a relative number of the total microparticles that comprise one or more of IL-1β, Ly6G, and CD66b.

4. The method of claim 1, further comprising determining in the biological sample a percentage of the total microparticles that comprise one of more of IL-1β, Ly6G, and CD66b.

5. The method of claim 4, wherein the IL-1β signature is indicated when the percentage of the total number of microparticles in the sample that comprise IL-1β is at least 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100%.

6-7. (canceled)

8. The method of claim 1, wherein the therapeutic regimen comprises administering to the subject confirmed to have the signature MP-associated disease or condition an effective amount of a gelsolin agent to treat the signature MP-associated disease or condition.

9. The method of claim 8, wherein administering the gelsolin agent has a greater therapeutic effect against the signature MP-associated disease or condition in the subject compared to a control therapeutic effect against the signature MP-associated disease or condition, optionally, wherein the control therapeutic effect is equal to an effect against the signature MP-associated disease or condition in a subject in the absence of administering the gelsolin agent.

10. (canceled)

11. The method of claim 1, wherein the signature MP-associated disease or condition is: hypoxia, decompression sickness, acute hypercarbia, chronic hypercarbia, sleep apnea, steroid-resistant asthma, or hypoxic ischemic encephalopathy, toxic gas toxicity, or asphyxiant gas toxicity.

12-13. (canceled)

14. The method of claim 1, wherein the signature MP-associated disease or condition is: a retinopathy, Alzheimer's disease, Multiple sclerosis, or a type 2 diabetes sequelae.

15. The method of claim 1, wherein the signature MP-associated disease or condition is one of: chronic obstructive pulmonary disease (COPD), chest wall deformity, a neuromuscular disease, obesity hypoventilation syndrome, respiratory failure, a hypoxia sequelae of a pneumonia, or acute severe asthma.

16. (canceled)

17. The method of claim 1, wherein the gelsolin agent comprises a gelsolin molecule, a functional fragment thereof, or a functional derivative of the gelsolin molecule, optionally wherein the gelsolin molecule is a plasma gelsolin (pGSN), and optionally wherein the gelsolin molecule is a recombinant gelsolin molecule.

18-20. (canceled)

21. The method of claim 1, wherein the administration of the gelsolin agent reduces severity of the signature MP-associated disease or condition in the subject by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100% compared to the severity of the signature MP-associated disease or condition of a control not administered the gelsolin agent.

22. The method of claim 1, further comprising, determining a level of severity of the signature MP-associated disease or condition in the subject, wherein a means of the determining comprises one or more of: an assay, observing the subject, assessing one or more physiological symptoms of the signature MP-associated disease or condition in the subject, assessing the history of the subject, and assessing a likelihood of survival of the subject.

23-29. (canceled)

30. The method of claim 1, wherein the subject is a mammal, and optionally is a human.

31. The method of claim 1, wherein the biological sample comprises a blood sample.

32. The method of claim 1, wherein the signature MP-associated disease or condition is not an infection.

33. The method of claim 1, wherein the signature MP-associated disease or condition is a post-infection sequelae.

34-36. (canceled)

37. A method for treating a signature MP-associated disease or condition in a subject, the method comprising, administering to a subject having or suspected of having the signature MP-associated disease or condition an effective amount of a gelsolin agent wherein the administered gelsolin agent has a greater therapeutic effect against the signature MP-associated disease or condition compared to a control therapeutic effect on the signature MP-associated disease or condition.

38-46. (canceled)

47. The method of claim 37, wherein the gelsolin agent comprises a gelsolin molecule, a functional fragment thereof, or a functional derivative of the gelsolin molecule, optionally wherein the gelsolin molecule is a plasma gelsolin (pGSN), and optionally wherein the gelsolin molecule is a recombinant gelsolin molecule.

48-69. (canceled)

70. A method for reducing a subject's risk of developing a signature MP-associated disease or condition, comprising: administering to a subject identified as at risk of developing the signature MP-associated disease or condition an effective amount of a gelsolin agent to reduce the subject's risk of developing the signature MP-associated disease or condition.

71-104. (canceled)
